(19) United States
(12) Patent Application Publication
(10) Pub. No.: US 2025/0179507 A1
(43) Pub. Date: Jun. 5, 2025

(54) Modulation of Protein Levels

(71) Applicant: Carlsberg A/S, Copenhagen V (DK)

(72) Inventors: Christian Poulsen, Albertslund (DK); Jesper Harholt, Copenhagen V (DK)

(73) Assignee: Carlsberg A/S, Copenhagen V (DK)

(21) Appl. No.: 18/844,546

(22) PCT Filed: Mar. 10, 2023
(86) PCT No.: PCT/EP2023/056176

§ 371 Date: Sep. 6, 2024

(30) Foreign Application Priority Data
Mar. 11, 2022 (EP) 22161674.1

(51) Int. Cl. C12N 15/67; C12N 15/10; C12N 15/82; C12Q 1/6827

(52) U.S. Cl. CPC C12N 15/67; C12N 15/102; C12N 15/82; C12Q 1/6827

(57) ABSTRACT

While eliminating a protein of interest in a eukaryotic organism is relatively straightforward, non-GMO methods based on nucleotide substitutions for modulating the translational efficiency and levels of a protein towards a pre-determined aim have proven difficult. This is due to the complexity of the protein expression machinery which makes it difficult to predict and elucidate the effects of a substitution in a nucleotide sequence on the modulation of an endogenous protein expression. The present invention relates to a method for modulating levels of a protein of interest in a eukaryotic organism of a species of interest, leading to increasing the probability of either higher or lower levels of the protein of interest. Further, the present invention relates to an eukaryotic organism comprising one or more mutation(s) associated with this modulation.

### TECHNICAL FIELD

The present invention provides technology to modulate translation efficiency of an endogenous protein of interest in an eukaryotic organism of a species of interest. The methods of the invention are for example useful for modulating the abundance of target protein, while preserving native functionality, including native spatial and/or temporal transcription profile, thereby reducing the risk of undesired pleiotropic effects.

### BACKGROUND

Genetic methods to generate genetically modified (GM) organisms (GMOs) are widely available. However, for many purposes, particularly in the food and beverage industries, use of GMOs is often less desirable. Conventional methods are available that allows identification of predetermined mutations in nucleotides of interest in a library of a traditionally mutagenized organism. Such libraries in general comprise mainly single nucleotide substitutions. However, deduction of which nucleotide substitution is likely to generate a desired effect, such as modulation of protein levels, requires extensive knowledge of the protein of interest. Eliminating a protein of interest may be relatively straightforward using modern identification methods and can be obtained by identifying a premature STOP codon in the gene coding sequence. In contrast, modulating abundance of protein of interest has, in many cases, proven to be difficult to achieve with a single nucleotide substitution. This is due to the extreme complexity of the molecular mechanics by which a gene constituted by a sequence of nucleotides is transcribed into messenger RNA and translated into a protein. Nonetheless, numerous factors affecting the speed of this process have been discovered and studied in detail including the role of transcription factor binding sites, cis-acting elements, hairpin loops, internal ribosomal entry sites (IRESs), microRNA binding sites etc. (Merchante et al., 2017, Ref. 1). Common for all these factors are that they diverge between genes both regarding nucleotide sequence but also regarding position and importance. Consequently, it is a difficult task to elucidate where in the nucleotide sequence of a gene of interest, a nucleotide substitution will modulate the gene expression towards a pre-determined aim.

### SUMMARY

Traditional approaches to alter protein of interest content in vivo has been to modify the transcript level. This approach is however not trivial and require substantial knowledge about cis-acting elements and typically also utilisation of GMO technology. Importantly, genes in eukaryotic organisms all share the translation initiation start ATG (AUG in mRNA). Studies have shown that specifically the nucleotides located immediate up and downstream of AUG have a pronounced effect on the translational efficiency on recombinant expression systems. For instance, Kim et al. 2014 (Ref. 3) and Agarwal et al. 2009 (Ref. 4) have used artificial constructs to study the impact of sequence diversity of the 5′UTRs of Arabidopsis genes in their translational efficiency and the expression of recombinant human a1-proteinase inhibitor in transgenic tomato plants respectively (Ref. 3). Gupta et al. (2016, Ref. 2) have studied in-silico the context sequence around the initiation codon in plants, and have noted a correlation between nucleotide bias and constitutive protein abundance in pools of 500 highly- and poorly expressed genes in Arabidopsis and Rice.

Modulation of endogenous protein activity is frequently of interest in order to induce beneficial protein activity or to reduce less desirable protein activity to lower levels. However, generally applicable strategies to obtain such modulation are not available.

Accordingly, novel methods for generating or isolating organisms with modulated endogenous protein levels in a more predictable manner in non-GMO variant organisms are thus highly needed.

The present invention combines single nucleotide polymorphism identification or generation technology, elucidation of a consensus matrix around start site, and altered translation initiation site to increase the probability of specifically regulating levels of an endogenous protein of interest. The technology platform comprises deciphering the translation initiation sequence for a given eukaryotic species to provide the relative frequencies of each nucleotide at each position in combination with methods and tools to alter said sequence in an endogenous gene of interest to modulate translational efficiency leading to altered level of said endogenous protein of interest content in vivo. Importantly, the invention allows modulating the levels of endogenous protein without need for any recombinant methods. Advantages include but are not limited to, exclusively changing the protein abundance but keeping native spatial-temporal expression profile preserving native functionality, and fine tuning protein abundance instead of generating knock out or utilising highly and/or ubiquitously expressed promotors, thereby reducing the risk of undesired pleiotropic effects. In particular, the claimed methods may be performed in a non-GMO manner.

A first aspect of the invention relates to a method for modulating levels of a protein of interest in a eukaryotic organism of a species of interest, or a method of identifying a eukaryotic organism of a species of interest having modulated levels of a protein of interest, said method comprising the steps of:
a) obtaining the gDNA sequence of the translation initiation sequence (TIS) of a gene of interest encoding the protein of interest of the species,
b) comparing the gDNA sequence of the TIS of said gene with the relative frequency of each nucleotide in one or more positions of the TIS, preferably in each position of the TIS in said species or a highly similar species,
c) generating a variant organism carrying mutation(s) or isolating a variant organism carrying mutation(s), wherein said mutation(s) is (are) substitution(s) of one or more nucleotide(s) in the TIS of the endogenous gene,
wherein a substitution to a nucleotide identified as having a higher relative frequency at that position increases the probability of higher levels of the protein of interest, and
wherein a substitution to a nucleotide identified as having a lower relative frequency at that position increases the probability of lower levels of the protein of interest, and
with the proviso that the eukaryotic organism is not human.

A second aspect of the invention relates to an eukaryotic organism comprising one or more mutation(s), wherein the mutation(s) is(are) in the translation initiation sequence (TIS) of a gene coding for a protein of interest, and wherein said mutation(s) is a(are) substitution(s) of one or more nucleotide(s) in the TIS of the endogenous gene,
wherein a substitution to a nucleotide identified as having a higher relative frequency at that position increases the probability of higher expression levels of the protein of interest, and wherein
a substitution to a nucleotide identified as having a lower relative frequency at that position increases the probability of lower expression levels of the protein of interest.

### DESCRIPTION OF DRAWINGS

FIG. 1: Down-regulation of grain specific β-glucanase

A) Schematic representation of the −3 C/T substitution identified in the barley variant, LIM-19. B) Graphical presentation of the β-glucanase enzymatic activity data found in Table 5 (in WT organism, grey bar and LIM-19 (C/T at pos. −3 from ATG), black bar),

FIG. 2: Up-regulation of Sucrose Transporter 2 in barley

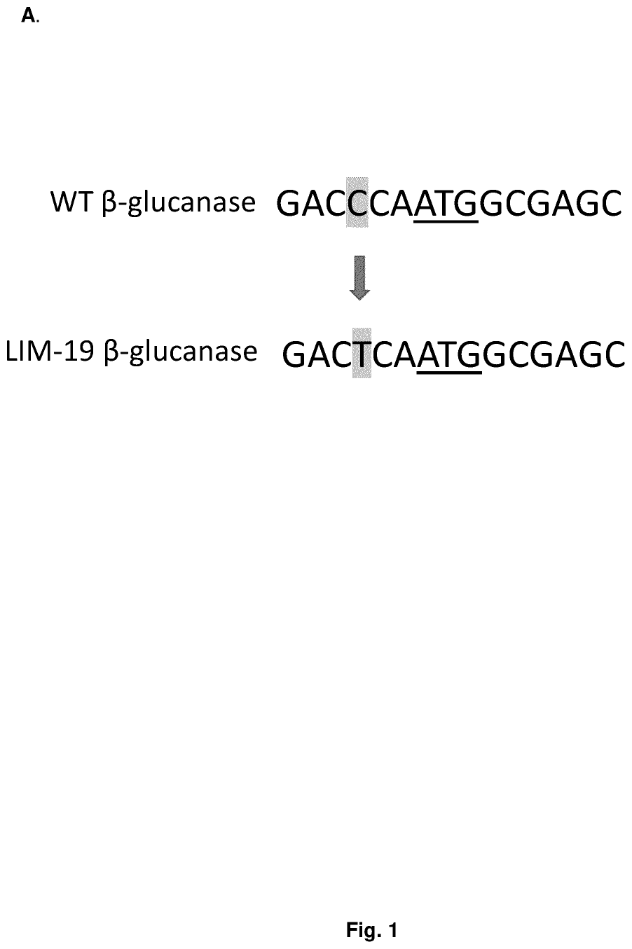

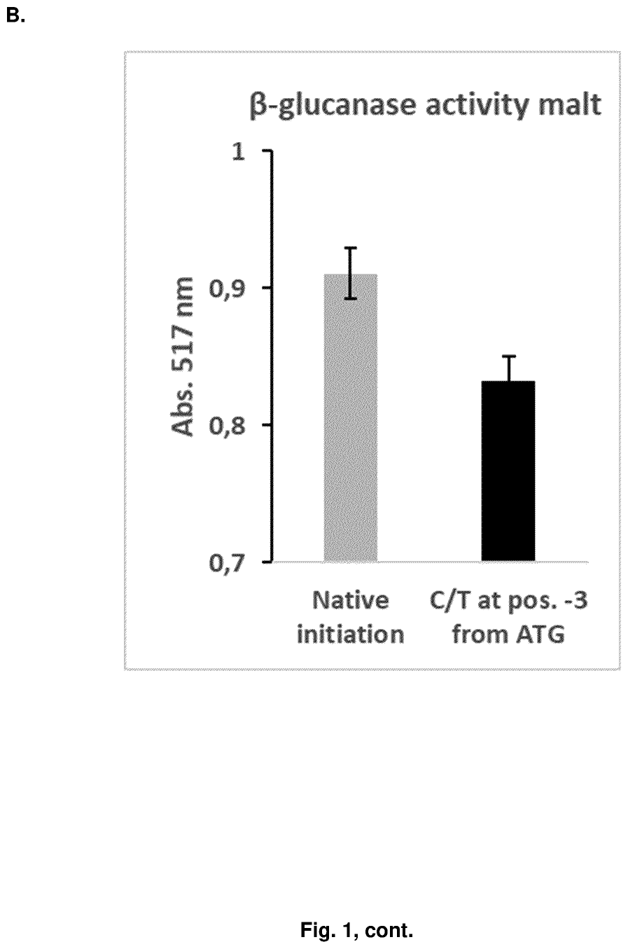

A) Schematic representation of the +4 C/G substitution identified in barley variant, LIM-14, B) Quantitative analysis of mRNA transcript encoding Sucrose Transporter 2 (SUT2) “Expression” in native (“WT”) and variant, LIM-14 (HO) (A sequence of the barley SUT2 gene is provided as SEQ ID NO:13), C) Quantitative analysis of SUT2 protein in native (“WT”) and variant, LIM-14 (HO), D) Protein vs. transcript ratio quantification in WT and LIM-14 (HO), E) Average barley grain weights distribution from 3 consecutive growing seasons from WT (dotted line) and LIM-14 (solid line) plants. Average of 490 grains weighed per year per genotype. The distribution was binned at 10 mg for illustration purpose.

FIG. 3: Schematic representation of the +4 (G/A) substitution identified in Aspergillus oryzae.

WT: wild-type, CS-Lo: Aspergillus oryzae citrate synthase+4 (G/A) variant.

FIG. 4: Quantification of Citric acid levels (ng/μl) at 24, 48, 72 and 96 H after fermentation in PD Broth through HPLC in WT and variant CS-Lo genotype.

T-test analysis at 48H showed t(4)=−3.3, p=0.03*. Citric acid levels in additional timepoints were below background levels detected in PD Broth indicating no production of Citric acid by both WT and CS-Lo strain. (NA: not applicable)

### DETAILED DESCRIPTION

### Definitions

The term “approximately” as used herein in relation to numbers refers to ±10%, preferably ±5%, for example to ±1%.

The term “barley” in reference to the process of making barley based beverages, such as beer, particularly when used to describe the malting process, means barley kernels. In all other cases, unless otherwise specified, “barley” means the barley plant (Hordeum vulgare, L.), including any breeding line or cultivar or variety, whereas part of a barley plant may be any part of a barley plant, for example an external plant structure such as leaves, stems, roots, flowers and grains, known as plant organs. It may also include any tissue or cells.

The term “β-glucan” as used herein, unless otherwise specified, refers to the plant cell wall polymer “(1,3;1,4)-β-glucan”.

The term “β-glucanase” as used herein refers to enzymes with the potential to depolymerize β-glucan. Accordingly, unless otherwise specified, the term “β-glucanase” refers to an endo- or exo-enzyme or mixture thereof characterized by (1,3;1,4)-β- and/or (1,4)-β-glucanase activity.

As used herein, the term “consensus matrix” refers to a matrix indicating the relative frequency of each nucleotide in each position of a TIS of the species of interest. The consensus matrix is constructed from data obtained analyzing the frequency of the nucleotides at each position of the translation initiation sequence across a large number of genes sequences of the species of interest, establishing for each nucleotide position the relative frequency of the presence of each of the fours nucleotides A, T, C or G at each position. Said large number is preferably at least 100, such as at least 1000, such as at least 5000, for example in range of 1000 to 30.000. The relative frequencies in the consensus matrix can be for instance be provided as a percentage, i.e. the calculated ratio per 100 observations, such as 100 nucleotide sequences or 100 genes, i.e. as a percentage. For each nucleotide position the nucleotides may be arranged in order of decreasing relative frequency.

As used herein, the term “consensus sequence” refers to the sequence composed of the succession of nucleotides each being the most relatively frequent at each nucleotide position of the TIS of the species of interest. The consensus sequence is established by analyzing the frequency of the nucleotides at each position of the translation initiation sequence across a large number of genes of the species of interest, establishing for each nucleotide position a probability of the presence of each of the nucleotide A, T, C or G at each position. Said large number is preferably at least 100, such as at least 1000, such as at least 5000, for example in range of 1000 to 30.000. As an example, the Kozak sequence or Kozak motif is typically recognized as the consensus sequence in Vertebrates. In some cases, the consensus sequence may be established integrating sequences from similar, closely related species, for instance if a limited number of sequences is available for the species of interest.

The term “dPCR” refers to digital polymerase chain reaction. It can be used to directly quantify and clonally amplify nucleic acids strands including DNA, cDNA or RNA. dPCR measures nucleic acids amounts in a more precise manner than PCR. In conventional PCR, one reaction is carried out per single sample. dPCR also relies on performing a single reaction within a sample, however the sample is compartmentalized, i.e. the sample is separated into a large number of partitions and the reaction is carried out in each partition individually.

The term “ddPCR” refers to droplet digital polymerase chain reaction. In ddPCR, one or more PCR amplifications are performed, wherein each reaction is separated into a plurality of water-oil emulsion droplets, so that PCR amplification of the target sequence may occur in each individual droplet. ddPCR is an example of a compartmentalized PCR.

The term “genotype” as used herein refers to an organism comprising a specific set of genes. Thus, two organism comprising identical genomes are of the same genotype. An organism's genotype in relation to a particular gene is determined by the alleles carried by said organism. In diploid organisms the genotype for a given gene may be AA (homozygous, dominant) or Aa (heterozygous) or aa (homozygous, recessive).

A sequence composed of the succession of nucleotides each being the least relatively frequent at each nucleotide position of the TIS in a given species may herein be referred to as “least frequent TIS” of said species.

As used herein, the nucleotide positions numbering is defined as the mRNA start codon, AUG (or ATG in the corresponding DNA sequence) having positions +1, +2 and +3 respectively. The nucleotide directly in 5′ of the AUG or ATG being numbered as −1 and the nucleotide directly in 3′ of the AUG or ATG codon being numbered as +4.

Nucleotides further upstream or downstream of the AUG or ATG codon are thus numbered with decreasing negative integers or increasing positive integers respectively. In some cases nucleotide positions are also indicated by their “base pair” or “bp” numbering, surrounding the start ATG (AUG) codon, using the same numbering as indicated above.

The term “parent organism” or “parent” means the corresponding organism from which a variant is derived, for instance from which a nucleotide substitution was generated or identified to isolate the variant organism, as described in the present invention. If the variant is generated by random mutagenesis of a given strain or variety of said species, the parent organism is said strain or variety, which has not been subjected to said random mutagenesis. The parent and variant organism may also for instance differ from each by other additional mutations not interfering with the modulation of the gene expression of the protein of interest, such as for instance different silent mutations.

The term “wild type” as used herein refers to the naturally occurring sequence of a nucleic acid at a genetic locus in the genome of an organism, and sequences transcribed or translated from such a nucleic acid. Thus, the term “wild-type” may also refer to the amino acid sequence encoded by the nucleic acid. In some cases the “parent organism” described herein may correspond to the “wild type” organism, i.e. that the parent sequence of a nucleic acid at a genetic locus in the genome of the parent organism, and sequences transcribed or translated from such a nucleic acid, may be in some cases wild type sequences.

The term “PCR” as used herein refers to a polymerase chain reaction. A PCR is a reaction for amplification of nucleic acids. The method relies on thermal cycling, and consists of cycles of repeated heating and cooling of the reaction to obtain sequential melting and enzymatic replication of said DNA. In the first step, the two strands forming the DNA double helix are physically separated at a high temperature in a process also known as DNA melting. In the second step, the temperature is lowered allowing enzymatic replication of DNA. PCR may also involve incubation at additional temperature in order to enhance annealing of primers and/or to optimise the temperature(s) for replication. In a PCR, the temperature generally cycles between the various temperatures for a number of cycles.

The term “PCR reagents” as used herein refers to reagents, which are added to a PCR in addition to a sample and a set of primers. The PCR reagents comprise at least nucleotides and a nucleic acid polymerase. In addition, the PCR reagents may comprise other compounds such as salt(s) and buffer(s).

The term “relative frequency”, or “Rel. freq.” as used herein, with respect to a nucleotide, refers to the calculated frequency of presence of a certain nucleotide at a specific position in a nucleotide sequence, of all 4 possible nucleotides. For instance the relative frequency can be the calculated frequency of presence of a certain nucleotide of all 4 possible nucleotides at each position of the TIS. The relative frequencies can be for instance be provided as a percentage, i.e. the calculated ratio per 100 observations, such as per 100 nucleotide sequences or 100 genes. The relative frequency may also be understood as the calculated probability of the presence of a nucleotide, for instance at a specific position in a nucleotide sequence, expressed as a percentage. Preferably, the relative frequency of nucleotides at each position of the TIS is determined based on at least 100 TIS sequences. Thus, for a given species, the relative frequency of nucleotides at each position of the TIS is determined based on at least 100 TIS sequences from said species.

The term “reproduction” as used herein refers to both sexual and asexual reproduction. Thus, reproduction may be multiplying an organism in a clonal manner (also known as “asexual reproduction”). Reproduction may also be generating progeny of an organism, wherein the progeny comprises allele(s) from the parent organism. Thus, reproduction of an organism comprising a mutant allele may refer to generating progeny of said organism, wherein the progeny comprises the mutant allele. Preferably, the mutant allele carries one or more of the mutation(s) in the NOI(s).

The term “reproductive parts of an organism” as used herein refers to any part of an organism which under the right conditions may grow into an entire organism. By way of example, in embodiments of the invention where the species is a plant, the reproductive part of said organism may, for example, be a seed, a grain or an embryo of said plant. In embodiments of the invention, wherein the species is a unicellular organism, then the reproductive parts is the entire organism, i.e. one cell.

The term “sensitive detection means” refers to detection means such that they allow detection of one mutant organism in a library of at least 300 organisms, even in cases where the mutant organism's genotype differs from the genotypes of the non-mutant organisms by at the most one nucleotide. Preferably they allow detection of one mutant genome in a library of 300 genomes.

The term “Translational efficiency”, as used herein, refers to the rate at which an mRNA is decoded to produce a specific polypeptide according to the rules specified by the genetic code.

As used herein, the term “translation initiation sequence”, or “translation initiation site” (TIS) refers to the nucleotide sequence region comprising the mRNA start codon, AUG (or ATG in the corresponding DNA sequence) that initiates translation in Eukaryotes, preferably comprising the region ranging from the −10 to +13 positions nucleotide positions around the mRNA AUG codon (or ATG in the corresponding DNA sequence), more preferably the −6 to +9 nucleotide positions around the mRNA AUG codon (or ATG in the corresponding DNA sequence). The translation initiation site (TIS) differs from gene to gene. The invention shows that specific nucleotides influence how efficient mRNA is translated into protein. The relation between nucleotide sequence and translation efficiency is species related, but with phylogenetically less diverge species sharing less diverse relation between nucleotide sequence of TIS and protein translation efficiency.

### Method for Modulating Levels of a Protein of Interest

A first aspect of the present invention relates to a method for modulating levels of a protein of interest in a eukaryotic organism of a species of interest (e.g. any of the organism described herein below in the section “Organism”, or a method of identifying a eukaryotic organism of a species of interest having modulated levels of a protein of interest, said method comprising the steps of:
a) Obtaining the gDNA sequence of the translation initiation sequence (TIS) of a gene of interest encoding the protein of interest of the organism. The TIS is the nucleotide sequence region comprising the start codon of a gene. Said sequence can be obtained by obtaining genomic sequence information of the gene of interest, e.g. from a database or by sequencing said gene.
b) Comparing the gDNA sequence of the TIS of said gene with the relative frequency of each nucleotide in in one or more positions of the TIS, preferably in each position of the TIS in said species or a highly similar species. In order to prepare said comparison, the relative frequency of each nucleotide in each position of the TIS is determined by comparing TIS of multiple genes of said species or a similar species. It is preferred that the relative frequency from the same species is used. In order to understand the relative frequencies of each nucleotide at each position of the TIS in a given species, a consensus matrix may be generated as described herein below in the section “Relative frequency and consensus matrix”.
c) Generating a variant organism carrying mutation(s) or isolating a variant organism carrying mutation(s), wherein said mutation(s) is (are) substitution(s) of one or more nucleotide(s) in the TIS of the endogenous gene. Said variant may e.g. be generated by random mutagenesis followed by isolation of the relevant variants using a method involving generation of sub-pools and sensitive detection methods as described herein below in the sections “Identification of variants”, “Organisation of sub-pools”, “Preparing DNA samples” and “Sensitive detection means”. In some embodiments, the single nucleotide polymorphism identification technology is using programmable nucleases, e.g. those described in the section “Identification of variants”.

In some embodiments, the organism of interest is a plant, and the step c) of generating or isolating a variant organism carrying mutation(s), further comprises a step of random mutagenesis.

In other embodiments wherein the organism of interest is a plant or an animal, the step c) of generating or isolating a variant organism carrying mutation(s) further comprises a step of gene editing using programmable nucleases, a CRISPR guide RNA system, a base editor, and/or a prime editor.

The mutation(s) is (are) substitution(s) of one or more nucleotide(s) in the TIS of the endogenous gene. In preferred embodiments, each variant carries only one mutation in the TIS. As explained herein, a substitution to a nucleotide identified as having a higher relative frequency in the consensus matrix of the species at that position increases the probability of higher expression levels of the protein of interest, and a substitution to a nucleotide identified as having a lower relative frequency in the consensus matrix of the species at that position increases the probability of lower expression levels of the protein of interest.

### Organism

As noted above, the invention relates to methods for modulating levels of a protein of interest in a eukaryotic organism of a species interest. Said organism may be any of the organisms described herein in this section.

In another aspect, the invention relates to a eukaryotic organism of a species of interest comprising one or more mutation(s), wherein the mutation(s) is(are) in the translation initiation sequence (TIS) of a gene coding for a protein of interest, and wherein said mutation(s) is (are) substitution(s) of one or more nucleotide(s) in the TIS of the endogenous gene, wherein a substitution to a nucleotide identified as having a higher relative frequency in the consensus matrix of the species at that position increases the probability of higher expression levels of the protein of interest, and wherein a substitution to a nucleotide identified as having a lower relative frequency in the consensus matrix of the species at that position increases the probability of lower expression levels of the protein of interest.

The organism may be any eukaryotic organism, although the organism is not an entire human being.

The organism may be a multicellular organism or a unicellular organism. The organisms may also be cells derived from a multicellular organism, e.g. a cell line.

In some embodiments, the organism is a fungus or a plant. Said plant may be a green plant, e.g. a plant selected from the group consisting of flowering plants, conifers, gymnosperms, ferns, clubmosses, hornworts, liverworts, mosses, green algae and brown algae.

In some embodiments, the fungus is a yeast or a filamentous fungus. The yeast may for example be a yeast of the genus Saccharomyces, such as S. cerevisiae or S. pastorianus. The filamentous fungus may for example be a filamentous fungus of the genera Aspergillus, Fusarium, Beauveria, or Metarhizium, for instance Aspergillus oryzae (Koji mold). In some embodiments, the organism is Aspergillus oryzae strain RIB40.

In some embodiments, the plant is selected from the group consisting of monocots or dicots.

In particular, the plant may be a domesticated plant. Said domesticated plant may be any plant cultivated by humans, e.g. as a source of food, feed or as a raw material for production of goods or for aesthetic purposes. In one preferred embodiment, the plant is a cereal. A “cereal”, as defined herein, is a member of the Graminae plant family, cultivated primarily for their starch-containing seeds or grains. Cereals include, but are not limited to, barley (Hordeum), wheat (Triticum), rice (Oryza), maize (Zea), rye (Secale), oat (Avena), sorghum (Sorghum) and the wheat-rye hybrid Triticale. The plant may also be other domesticated plants, including tomato.

In some embodiments, the monocot or dicot is selected from the group consisting of barley, rice, wheat, corn, oat, peas, yellow peas, chickpeas, faba beans, rapeseed, durum wheat, risotto rice, bitter gourd, quinoa, alfalfa, lupin and soy. Examples 1 and 2 below are for instance performed in barley.

In some embodiments, wherein the organisms of the present invention are plants or animals, the plant or animal is not exclusively obtained by means of an essentially biological process (EBP).

In another aspect, the invention relates to an organism prepared by the method of the present invention.

### Identification of Variants

In some embodiments, the variant organism comprising the substitution(s) is isolated using a single nucleotide polymorphism (SNP) identification technology. Variants may also be referred to as “mutants” herein.

The person skilled in the art will appreciate that SNP identification technology include for example hybridization-based methods, such as microarrays, methods involving molecular beacons e.g. fluorescent DNA probes containing a fluorophore and a quencher, enzyme-based methods, such as methods involving PCR amplification.

In some embodiments, the single nucleotide polymorphism identification technology is using programmable nucleases. The skilled person will appreciate that one approach may be to use nucleases programmed using a guide RNA, for instance a guide RNA complementary to a specific target DNA sequence, such as the sequence containing the single nucleotide polymorphism(s) e.g. the substitution(s) generated or isolated in the variant organism.

In some embodiments, the single nucleotide polymorphism identification technology is using a CRISPR guide RNA system. The CRISPR guide RNA system may be a CRISPR-cas system. The skilled person will appreciate that several technologies and platforms have been developed based on for instance cas9, cas12, cas13 or cas14 nucleases, for DNA detection, such as for instance the SHERLOCK platform (Gootenberg et al., 2017 (Ref. 8)).

In some embodiments, the single nucleotide polymorphism identification technology is using a base editor or a prime editor. The person skilled in the art will appreciate the applications of the CRISPR system for base editing and prime editing, for instance to facilitate single nucleotide substitutions. Useful examples and guidance on base editors and prime editors in plant can for example be found in Molla et al. 2021 (Ref. 9).

The single nucleotide polymorphism identification technology can preferably be based on the method as described in WO2018001884A1 or the FindIT method described in Knudsen et al. 2021 (Ref. 10), both which are hereby incorporated by reference in their entirety. For example, the organism comprising said mutation(s) may be identified using as described in the below embodiments.

In some embodiments, the single nucleotide polymorphism identification technology comprises the steps of:
a. providing a pool comprising a plurality of organisms of the species of interest, or reproductive parts thereof, representing a plurality of different genotypes;
b. dividing said pool into one or more sub-pools of organisms, or reproductive parts thereof, wherein each sub-pool comprises more than one copy of organisms of each genotype or reproductive parts thereof;
c. obtaining at least two random fractions of said sub-pool, wherein said fractions in theory each comprises organisms representing each genotype of said sub-pool
d. preparing gDNA samples from one fraction of each sub-pool, while maintaining the at least one fraction of each sub-pool for potential multiplication of organisms of each genotype within said sub-pool;
e. detecting said substitution(s) in said gDNA samples, thereby identifying sub-pool(s) comprising organism(s) or reproductive parts thereof comprising said substitution(s)
f. identifying from said identified sub-pool one or more individual organisms comprising said substitution(s).

In some embodiments, the pool of organisms comprises at least 10,000, preferably at least 100,000, yet more preferably at least 500,000 organisms, or reproductive parts thereof, with different genotypes.

In some embodiments, the pool of organisms is generated by subjecting a plurality of organisms of reproductive parts thereof to a step of random mutagenesis.

In some embodiments, at least 10,000, preferably at least 100,000, yet more preferably at least 500,000 organisms, or reproductive parts thereof, are subjected to random mutagenesis.

In embodiments of the invention, wherein the organism is a plant, the pool may be as described in the section “Pool” on p. 22-28 of international patent application WO 2021/069614. Thus, the size of the pool may in such embodiments be in the range of 0.7 to 1.3× the optimal library size, wherein the optimal library size is determined as described in the section “Pool” on p. 22-28 on p. 22-28 of international patent application WO 2021/069614.

In some embodiments, the methods comprise a step of reproduction of the organisms, or reproductive parts thereof, within the pool, and said step of reproducing may be performed simultaneously with, or subsequent to the step of dividing the organisms into sub-pools.

In some embodiments, the organism is a plant, and all seeds of a given plant are placed into the same sub-pool.

The division of pools into sub-pools can be performed for instance as described in the section “Dividing a pool of regenerative parts into sub-pools” of WO2018001884A1 or as in the FindIT method described in Knudsen et al. 2021 (Ref. 10) or as described below in the section “Organisation of sub-pools”.

### Organisation of Sub-Pools

The organisation of the sub-pools can be performed for instance as described in WO2018001884A1 or the FindIT method described in Knudsen et al. 2021 (Ref. 10), Prior to the step of identifying sub-pools, the method may also comprise a step of dividing the pool of organism into sub-pools and organising said sub-pools in a manner, which eases the identification of a sub-pool comprising organisms, or reproductive parts thereof comprising the mutation of interest.

For example, fractions of DNA samples from several sub-pools may be combined into “Super-pools”. Super-pools comprising DNA comprising the mutation of interest may then be identified using extremely sensitive detection means. Afterwards only sub-pools comprised in super-pools, comprising the DNA with the mutation of interest must be tested. Useful methods for preparing and testing super-pools are described in international patent application WO 2018/001884.

In another example sub-pools are placed into multi-dimensional grids, and super-pools are prepared of the sub-pools in each dimension. This is easiest explained using a 2 dimensional grid as example. Each sub-pool given a coordinate (x,y) in said grid. Fractions of DNA samples of all sub-pools with a given coordinate X are pooled and tested for the presence of DNA comprising the mutation of interest. Similarly, fractions of DNA samples of all sub-pools with a given coordinate Y are pooled and tested for the presence of DNA comprising the mutation of interest. Once super-pools comprising DNA samples from a given coordinate X and a given coordinate Y are identified as comprising DNA with the mutation of interest, a sub-pool comprising said DNA can be identified based on its coordinates X and Y. The skilled person will understand that a similar system can be made using multiple dimensions, rather than only two. Thus, rather than testing each sub-pool for the presence of DNA comprising the mutation of interest it is also comprised within the methods of the invention, that the sub-pools are organised so that groups of sub-pools can be tested together. The testing may for example be done using any of the sensitive detection means described herein.

Usually, the pool is divided into a plurality of sub-pools, preferably into at least 2 sub-pools, such as into at least 50 sub-pools, such as into at least 100 sub-pools, preferably into at least 200 sub-pools, more preferably into at least 300 sub-pools, even more preferably into at least 400 sub-pools, yet more preferably into at least 500 sub-pools, even more preferably into at least 1000 sub-pools, yet more preferably into at least 1500 sub-pools. There is, in principle, no upper limit to the number of sub-pools. Typically, however, the pool is divided into at the most 50,000, such as at the most 25,000, e.g. at the most 10,000 sub-pools, for example at the most 2000.

Each sub-pool preferably comprises a plurality of organisms or reproductive parts thereof, representing a plurality of genotypes. Preferably, each sub-pool comprises at least 10, more preferably at least 100, yet more preferably at least 150, even more preferably at least 200, even more preferably at least 300, even more preferably at least 400, even more preferably at least 500, yet more preferably at least 1,000, even more preferably at least 5,000 organisms or reproductive parts thereof, with different genotypes. In some embodiments, each sub-pool comprises at the most 5,000, such as at the most 2000 organisms or reproductive parts thereof, with different genotypes. For example, each sub-pool may comprise in the range of 50 to 5000, such as in the range of 100 to 5000, for example in the range of 150 to 5000 organisms, or reproductive parts thereof, with different genotypes.

In embodiments of the invention where the organism is a plant, the pool may be divided into sub-pools as described in the section “Dividing a pool of regenerative parts into sub-pools” on p. 32-36 of international patent application WO 2021/069614. Thus, in such embodiments, the sub-pool may comprise in the range of 0.7 to 1× the maximum sub-pool size as calculated as described in WO 2021/069614 on p. 33-34.

### Preparing DNA Samples

The preparation of DNA samples can be performed for instance as described in the “Preparing DNA samples” section of WO2018001884A1 or as in the FindIT method described in Knudsen et al. 2021 (Ref. 10).

Preferably, the DNA samples are prepared by a method comprising the steps of (1) dividing each sub-pool into random fractions; (2) preparing DNA samples from an entire fraction.

Preferably the fractions of each sub-pool are prepared in a manner so that at least two fractions in theory comprises organisms, or reproductive parts thereof of all the genotypes represented in the sub-pool. Preferably, all fractions in theory comprises organisms, or reproductive parts thereof of all the genotypes represented in the sub-pool. This may for example be accomplished by mixing all organisms, or reproductive parts thereof of a sub-pool well, randomly dividing the sub-pool into in the range of 2 to 10 fractions, such as into in the range of 2 to 6 fractions, for example into in the range of 2 to 4 fractions. It is preferred that said at least 2 fractions each comprises in the range of 10 to 50%, such as in the range of 15 to 50%, for example in the range of 20 to 50% of the organisms, or reproductive parts thereof of the sub-pool.

Thus, the present methods do not require that DNA samples are prepared individually for each of the organisms or reproductive parts thereof comprised within one fraction. In fact it is preferred that an entire fraction is used to obtain a DNA sample, which in practice is a pooled DNA sample comprising DNA from all the genotypes comprised within the fraction. The DNA sample may be a gDNA sample or it may be cDNA, for example a cDNA sample prepared from an mRNA sample as is known in the art. Thus, preferably the methods do not comprise the step of preparing individual DNA samples for each plant or regenerative part of one fraction or sub-pool.

The methods of the invention comprise one or more steps of preparing DNA samples, in particular gDNA or cDNA samples prepared from mRNA samples, preferably gDNA samples. In particular, the methods may comprise one step of preparing DNA samples from an entire fraction.

The methods of the invention may also comprise a step of preparing DNA samples from a secondary sub-pool.

In general, said DNA samples of sub-pools are prepared in a manner so that the DNA sample in theory comprises DNA from each genotype within a sub-pool. The DNA sample may be prepared from an entire fraction, while the potential for reproduction of the organisms, or reproductive parts thereof of each genotype are maintained in the other fraction(s) of the sub-pool.

Thus, each sub-pool in general comprises more than one individual organism, or reproductive part thereof of each genotype, so that when divided in fractions, each fraction comprises more than one individual organism, or reproductive part thereof of each genotype. In particular, it may be preferred that each sub-pool comprises a sufficient amount of organisms, or reproductive parts thereof of each genotype in order to be able to randomly divide the sub-pool or the super-pool into in the range of 2 to 10 fractions, such as in the range of 2 to 6 fractions, for example into 2, 3, or 4 fractions in a manner such that each part in theory comprises organisms, or reproductive parts thereof representing each genotype of the sub-pool.

In this manner, a part of the organisms, or reproductive parts thereof from the sub-pool, e.g. in the range of 10 to 90%, preferably in the range 10 to 50%, such as in the range of 15 to 50%, for example in the range of 20 to 50% of the organisms, or reproductive parts thereof of each sub-pool may be used for preparing the DNA sample. The remainder of the organisms, or reproductive parts thereof of the sub-pool may be stored under conditions maintaining the reproductive potential of said organisms, or reproductive parts thereof. In embodiments where the organism is a plant, it may be sufficient to store seeds of said plants. Seeds, e.g. cereal grains, may frequently be stored in any dry and dark place.

Whereas the sub-pool preferably comprise several individual organisms, or reproductive parts thereof of each genotype as noted herein elsewhere, then the secondary sub-pool frequently may comprise only a few—sometimes even only one—individual organisms, or reproductive parts thereof of each genotype. This is in particular the case, in embodiments where the organism is a plant.

As explained above, when obtaining DNA from sub-pools, typically the DNA is extracted from entire fractions of the sub-pools. However, when obtaining DNA from the secondary sub-pools, said DNA may also be obtained from samples from individual organisms, or reproductive parts thereof. When samples are obtained from individual organisms, or reproductive parts thereof it may be preferred that the samples are obtained in a manner not significantly impairing said organism, or reproductive parts thereof, with respect to the potential for reproduction. Thus, the DNA sample may be prepared from a sample comprising or consisting of a part of the organism, or reproductive part thereof that is not essential for reproduction. The sample may be obtained in any useful manner depending on the species, e.g. by using biopsy, cutting, drilling, grating, tearing or by applying a syringe equipped with a needle.

Once either the fraction, or the sample obtained from the organism, or reproductive part thereof, is available (herein also collectively referred to as “sample”), the DNA sample may be prepared from said samples in any useful manner. If said samples contain large structures, e.g. entire seeds, the first step for preparing a DNA sample such as a gDNA sample will typically involve dividing said contents of said sample into smaller parts, for example, by physical means, e.g. by crushing or milling. Methods of preparing the DNA sample typically comprise the steps of disrupting cells and/or tissues, e.g. by detergent, by enzymes (e.g. lyticase), by ultrasound or by combinations thereof—thereby creating a crude lysate. Said lysate may be separated from any remaining debris by any useful means. The crude lysate may constitute the DNA sample comprising a gDNA sample. Alternatively, the DNA, e.g. the gDNA or mRNA to be used to obtain a cDNA sample, may be further purified, e.g. by separating said DNA from the remainder of the lysate, e.g. by binding to a selective matrix, centrifugation, gradient centrifugation and/or precipitation (e.g. using a precipitating agent, such as a salt, an alcohol or magnetic beads). Prior to such separation, other components of the lysate—including proteins and/or nucleoproteins—may be denatured or destroyed, e.g. by use of enzymes and/or denaturing agents. Other RNA-containing molecules may be removed, e.g. with the aid of enzymes. Useful methods for preparing DNA samples such as cDNA samples or gDNA samples are, for example, described in Sambrook et al., Molecular Cloning—Laboratory Manual, ISBN 978-1-936113-42-2 (Ref. 15).

In some embodiments, the step of detecting said substitution(s) in gDNA samples is performed by sequencing-based technology. The person skilled in the art will appreciate that the sequencing-based technology may preferably be a high-throughput, high resolution sequencing technology, next generation sequencing (NGS), and may include technologies based on liquid or solid phase DNA amplification.

In some embodiments, the step of detecting substitution(s) in gDNA samples comprises:
i. performing a plurality of PCR amplifications, each comprising the gDNA sample from one sub-pool, wherein each PCR amplification comprises a plurality of compartmentalised PCR amplifications, each comprising part of said gDNA sample, one or more set(s) of primers each set flanking a target sequence comprising the TIS of the gene of interest encoding the protein of interest of the species and PCR reagents, thereby amplifying the target sequence(s);
ii. detecting PCR amplification product(s) comprising one or more target sequence(s) comprising said substitution(s), thereby identifying sub-pool(s) comprising organism(s) or reproductive parts thereof comprising said substitution(s);

Regardless of whether said PCR comprises compartmentalised PCR amplifications or not, then the PCR amplifications will in general comprise a gDNA sample, a set of primers flanking the target sequence and PCR reagents. Said PCR reagents may be any of the PCR reagents described herein in this section.

The PCR reagents, in general, comprise at least nucleotides and a nucleic acid polymerase. The nucleotides may be deoxy-ribonucleotide triphosphate molecules, and preferably the PCR reagents comprise at least dATP, dCTP, dGTP and dTTP. In some cases, the PCR reagents also comprise dUTP.

The nucleic acid polymerase may be any enzyme capable of catalysing template-dependent polymerisation of nucleotides, i.e. replication. The nucleic acid polymerase should tolerate the temperatures used for the PCR amplification, and it should have catalytic activity at the elongation temperature. Several thermostable nucleic acid polymerases are known to the skilled person. In some embodiments of the invention, the nucleic acid polymerase has 5′-3′ nuclease activity and can thus be used in the amplification reaction with a TaqMan® probe.

The nucleic acid polymerase may be Escherichia coli DNA polymerase I. The nucleic acid polymerase may also be Taq DNA polymerase, which has a DNA synthesis-dependent strand replacing 5′-3′ exonuclease activity. Other polymerases having 5′-3′ nuclease activity include, but are not limited to, rTth DNA polymerase. The Taq DNA polymerase, e.g. obtained from New England Biolabs, can include Crimon LongAmp® Taq DNA polymerase, Crimson Taq DNA Polymerase, Hemo KlenTaq™, or LongAmp® Taq.

In some cases, the nucleic acid polymerase can be, e.g. E. coli DNA polymerase, Klenow fragment of E. coli DNA polymerase I, T7 DNA polymerase, T4 DNA polymerase, Taq polymerase, Pfu DNA polymerase, Vent DNA polymerase, bacteriophage 29, REDTaq™, Genomic DNA polymerase, or Sequenase. DNA polymerases are described, e.g. in U.S. Patent Application Publication No. 20120258501.

In addition, the PCR reagents may comprise salts, buffers and detection means. The buffer may be any useful buffer, e.g. TRIS. The salt may be any useful salt, e.g. potassium chloride, magnesium chloride or magnesium acetate or magnesium sulfate. The PCR reagents may comprise a non-specific blocking agent, such as BSA, gelatin from bovine skin, beta-lactoglobulin, casein, dry milk, salmon sperm DNA or other common blocking agents.

The PCR reagents may also comprise bio-preservatives (e.g. NaN3), PCR enhancers (e.g. betaine, trehalose, etc.) and inhibitors (e.g. RNase inhibitors). Other additives can include dimethyl sulfoxide (DMSO), glycerol, betaine (mono)-hydrate, trehalose, 7-deaza-2′-deoxyguanosine triphosphate (7-deaza-2′-dGTP), bovine serum albumin (BSA), formamide (methanamide), tetramethylammonium chloride (TMAC), other tetraalkylammonium derivaties [e.g. tetraethyammonium chloride (TEA-CI)]; tetrapropylammonium chloride (TPrA-Cl) or non-ionic detergent, e.g. Triton X-100, Tween 20, Nonidet P-40 (NP-40) or PREXCEL-Q. Furthermore, the PCR reagents may also comprise one or more means for detection of PCR amplification product(s) comprising the mutation(s) in the NOI(s). Said means may be any detectable means, and they may be added as individual compounds or be associated with, or even covalently linked to, one of the primers. Detectable means include, but are not limited to, dyes, radioactive compounds, bioluminescent and fluorescent compounds. In a preferred embodiment, the means for detection is one or more probes.

In some embodiments, the plurality of PCR amplification(s) of the method to detect substitution(s) in gDNA samples is (are) performed by a method comprising the following steps:
a. preparing one or more PCR amplifications comprising the gDNA sample, one or more set(s) of primers each set flanking a target sequence and PCR reagents;
b. partitioning said PCR amplification(s) into a plurality of spatially separated compartments;
c. performing PCR amplification(s);
d. detecting PCR amplification products,
   and said spatially separated compartments for example are droplets, such as a water oil emulsion droplets, wherein each droplet for example has an average volume in the range of 0.1 to 10 nL, and/or each PCR for example is compartmentalized into in the range of 1000 to 100,000 spatially separated compartments.

In some embodiments, the PCR reagents of the method comprises
a. one or more mutation detection probes, wherein each mutation detection probe(s) comprise(s) an oligonucleotide optionally linked to detectable means, wherein the oligonucleotide is identical to—or complementary to—a target sequence, including a predetermined substitution of the TIS of the gene of interest; and/or
b. one or more reference detection probe(s), wherein each reference detection probe(s) comprise(s) an oligonucleotide optionally linked to detectable means, wherein the oligonucleotide is identical to—or complementary to—a target sequence, including a reference TIS of the gene of interest;
   and the mutant detection probe(s) optionally is (are) linked to a fluorophore and a quencher, and/or the reference detection probe optionally is linked to a different fluorophore and a quencher.

### Sensitive Detection of Mutations

The detection of mutations can be performed for instance as described in the section “Sensitive detection means” of the WO2018001884A1 or as in the FindIT method described in Knudsen et al. 2021 (Ref. 10).

The entire PCR reaction comprising a plurality of compartmentalised PCR amplifications may be prepared in a number of different manners. In one embodiment, the PCR amplification comprising a plurality of compartmentalised PCR amplifications may be conducted as a digital PCR (dPCR) amplification. Any dPCR amplification known to the skilled person may be used with the invention. In general, at least one dPCR amplification comprising a plurality of compartmentalised PCR amplifications will be prepared for each DNA sample prepared. Thus, at least one dPCR amplification comprising a plurality of compartmentalised dPCR amplifications will be prepared per fraction of each sub-pool.

In general, the compartmentalised dPCR amplification is prepared in a method comprising the steps of:
Preparing a dPCR amplification comprising the DNA sample, a set of primers flanking the target sequence and PCR reagents;
Partitioning said dPCR amplification so that nucleic acid molecules within the sample are localized and concentrated within a plurality of spatially separated compartments;
Performing a dPCR amplification;
Detecting dPCR-based amplification products.

The DNA sample may a gDNA sample or a cDNA sample obtained from an mRNA sample. In some embodiments, the DNA sample is a gDNA sample.

Said separated compartments may be any separate compartments in which PCR amplifications can be performed. For example, it may be well of a plate, e.g. a well of a micro well plate or a microtiter plate, it may be microfluidic chambers, it may be capillaries, it may be the dispersed phase of an emulsion, or it may be a droplet or miniaturized chambers of an array of miniaturized chambers. The separated compartments may also be discrete spots on a solid support, e.g. discrete nucleic acid binding surfaces.

It is generally preferred that the DNA sample is distributed randomly into the samples for compartmentalised dPCR amplification. It is also preferred that each compartmentalised dPCR amplification only comprises a small number of nucleic acids comprising the target sequence. Due to the nature of random distribution, there may be some variation in the number of nucleic acid molecules comprised in each compartmentalised dPCR amplification. In one embodiment, each compartmentalised dPCR amplification comprises, in average, at the most 10, such as at the most 5 nucleic acid molecules comprising the target sequence.

In one preferred embodiment of the invention, the dPCR amplification comprising a plurality of compartmentalised PCR amplification is a droplet digital polymerase chain reaction (ddPCR). ddPCR is a method to perform dPCR that is based on water-oil emulsion droplet technology. The PCR amplification is fractionated into a plurality of micro-droplets, and PCR amplification of the target sequence occurs in each individual droplet. In general, ddPCR technology uses PCR reagents and work flows similar to those used for performing conventional PCRs. Thus, the partitioning of the PCR amplification is a key aspect of the ddPCR technique.

Accordingly, compartmentalised ddPCR amplifications may be contained in droplets, which may, for example, include emulsion compositions, or mixtures of two or more immiscible fluids (for example as described in U.S. Pat. No. 7,622,280 or as described in the examples herein below). The droplets can be generated by devices described in WO/2010/036352. In particular, the droplets can be prepared using a droplet generator, for example QX200 Droplet Generator available from Bio-Rad Laboratories, USA (hereinafter abbreviated Bio-Rad). The term emulsion, as used herein, can refer to a mixture of immiscible liquids (such as oil and water). The emulsions may, for example, be water in oil droplets, e.g. as described in Hindson et al. (2011).The emulsions can thus comprise aqueous droplets within a continuous oil phase. The emulsions can also be oil-in-water emulsions, wherein the droplets are oil droplets within a continuous aqueous phase. The droplets used herein are normally designed to prevent mixing between compartments, with the content of an individual compartment not only being protected from evaporation, but also from coalescing with the contents of other compartments. Thus, each droplet can be regarded as a spatially separated compartment.

Each droplet for ddPCR may have any useful volume. Preferably, however, the droplets have a volume in the nL-range. Accordingly, it is preferred that the droplet volume, on average, is in the range of 0.1 to 10 nL.

Microfluidic methods of producing emulsion droplets using microchannel cross-flow focusing or physical agitation are known to produce either monodisperse or polydisperse emulsions. The droplets can be monodisperse droplets. Also, the droplets can be generated such that their sizes do not vary by more than ±5% of the average size of the droplets. In some cases, the droplets are generated such that the droplet sizes only vary with ±2% of the average size of droplets.

Higher mechanical stability can be useful for microfluidic manipulations and higher-shear fluidic processing (e.g. in microfluidic capillaries or through 90° turns, such as valves in fluidic paths). Pre- and post-thermally treated droplets, or capsules, can be mechanically stable to standard pipet manipulations and centrifugation.

A droplet can be formed by flowing an oil phase through an aqueous sample. The aqueous phase can comprise, or consist, of components in a PCR amplification, e.g. a PCR amplification comprising the DNA sample, a set of primers flanking the target sequence and PCR reagents, such as any of the PCR reagents described herein below in the section “PCR reagents”.

The oil phase can comprise a fluorinated base oil, which can be additionally stabilized by combination with a fluorinated surfactant such as a perfluorinated polyether. In some cases, the base oil can be one or more of HFE 7500, FC-40, FC-43, FC-70, or other common fluorinated oil. In some cases, the anionic surfactant is Ammonium Krytox (Krytox-AM), the ammonium salt of Krytox FSH, or morpholino derivative of Krytox-FSH.

The oil phase can further comprise an additive for tuning the oil properties, such as vapor pressure, viscosity or surface tension. Non-limiting examples include perfluoro-octanol and 1H,1H,2H,2H-Perfluorodecanol. The oil phase may also be a droplet generating oil, e.g. the Droplet Generation Oil available from Bio-Rad.

The emulsion can formulated to produce highly mono-disperse droplets having a liquid-like interfacial film that can be converted by heating into micro-capsules having a solid-like interfacial film; such micro-capsules can behave as bioreactors that retain their contents through a reaction process, such as PCR amplification. The conversion to microcapsule form can occur upon heating. For example, such conversion can occur at a temperature of greater than 50, 60, 70, 80, 90, or 95° C. In some cases, this heating occurs using a thermocycler. During the heating process, a fluid or mineral oil overlay can be used to prevent evaporation.

In some cases, the droplet is generated using a commercially available droplet generator, such as Bio-Rad QX100™ Droplet Generator or Bio-Rad QX200™ Droplet Generator. The ddPCR and subsequent detection may be carried out using a commercially available droplet reader, such as Bio-Rad QX100 or QX200™ Droplet Reader.

Each PCR amplification can be compartmentalized into any suitable number of compartments. However, in one preferred embodiment, each PCR amplification is compartmentalized into in the range of 1,000 to 100,000 compartments (e.g. droplets). For example, each PCR amplification may be compartmentalized into in the range of 10,000 to 50,000 compartments (e.g. droplets). For example, each PCR amplification may be compartmentalized into in the range of 15,000 to 25,000 compartments (e.g. droplets). Further, each PCR amplification may be compartmentalized into approximately 20,000 compartments (e.g. droplets).

In some embodiments of the method of the present invention wherein the organism is a plant or an animal, the method does not comprise a step of sexual reproduction.

### Relative Frequency and Consensus Matrix

The methods disclosed herein relates to a method comprising identifying or generating variant organisms wherein one or more nucleotides of the TIS of a gene of interest have been substituted. Said substitution is preferably to either a nucleotide with a lower relative frequency (for increasing the probability of lower expression levels) or to a nucleotide with a higher relative frequency (for increasing the probability of higher expression levels). In order to understand the relative frequencies of each nucleotide at each position of the TIS in a given species, a consensus matrix may be generated. It is not a requirement that a consensus matrix is generated, information of the relative frequencies of each nucleotide at each position of the TIS in a given species may also be obtained and organised in other ways.

The consensus matrix is a matrix indicating the relative frequency of each nucleotide in each position of TIS of the species of interest.

The consensus matrix is constructed from the data obtained analyzing the frequency of the nucleotides at each position of the translation initiation sequence across a large number of genes sequences of the species of interest, establishing for each nucleotide position the relative frequency of the presence of each of the nucleotide A, T, C or G at each position. Said large number is preferably at least 100, such as at least 1000, such as at least 5000, for example in range of 1000 to 50.000. In some embodiments, the consensus matrix is obtained by analyzing the TIS of more than 5000, preferably more than 10000, even more preferably more than 15000 genes of the species of interest, determining the relative frequency of each nucleotide A, T, G, and C at each nucleotide position of the TIS around the ATG start codon.

The relative frequencies in the consensus matrix can be for instance indicated as a ratio of 100 observations, such as 100 nucleotide sequences or 100 genes, i.e. as a percentage.

In some embodiments of the current invention, the consensus matrix can be represented as follows:

TABLE 1
General format example of the consensus matrix.

| Species | | | | |
|---|---|---|---|---|
| Position | Nucleotide relative frequencies | | | |
| −6 | Nucleotide 1 | Nucleotide 2 | Nucleotide 3 | Nucleotide 4 |
| | Rel. freq. | Rel. freq. | Rel. freq. | Rel. freq. |
| −5 | Nucleotide 1 | Nucleotide 2 | Nucleotide 3 | Nucleotide 4 |
| | Rel. freq. | Rel. freq. | Rel. freq. | Rel. freq. |
| −4 | Nucleotide 1 | Nucleotide 2 | Nucleotide 3 | Nucleotide 4 |
| | Rel. freq. | Rel. freq. | Rel. freq. | Rel. freq. |
| −3 | Nucleotide 1 | Nucleotide 2 | Nucleotide 3 | Nucleotide 4 |
| | Rel. freq. | Rel. freq. | Rel. freq. | Rel. freq. |
| −2 | Nucleotide 1 | Nucleotide 2 | Nucleotide 3 | Nucleotide 4 |
| | Rel. freq. | Rel. freq. | Rel. freq. | Rel. freq. |
| −1 | Nucleotide 1 | Nucleotide 2 | Nucleotide 3 | Nucleotide 4 |
| | Rel. freq. | Rel. freq. | Rel. freq. | Rel. freq. |
| 4 | Nucleotide 1 | Nucleotide 2 | Nucleotide 3 | Nucleotide 4 |
| | Rel. freq. | Rel. freq. | Rel. freq. | Rel. freq. |
| 5 | Nucleotide 1 | Nucleotide 2 | Nucleotide 3 | Nucleotide 4 |
| | Rel. freq. | Rel. freq. | Rel. freq. | Rel. freq. |
| 6 | Nucleotide 1 | Nucleotide 2 | Nucleotide 3 | Nucleotide 4 |
| | Rel. freq. | Rel. freq. | Rel. freq. | Rel. freq. |
| 7 | Nucleotide 1 | Nucleotide 2 | Nucleotide 3 | Nucleotide 4 |
| | Rel. freq. | Rel. freq. | Rel. freq. | Rel. freq. |
| 8 | Nucleotide 1 | Nucleotide 2 | Nucleotide 3 | Nucleotide 4 |
| | Rel. freq. | Rel. freq. | Rel. freq. | Rel. freq. |
| 9 | Nucleotide 1 | Nucleotide 2 | Nucleotide 3 | Nucleotide 4 |
| | Rel. freq. | Rel. freq. | Rel. freq. | Rel. freq. |

For each position, the nucleotides may be arranged in order of decreasing relative frequency, for instance from left to right.

As explained above, in order to calculate the relative frequencies and/or to prepare the consensus matrix, a high number of sequences of TIS of the species are retrieved and the relative frequencies of each nucleotide at each position are calculated. Said sequences may e.g. be retrieved from databases of genomic sequences or by sequencing relevant genes or by a combination of both. The relative frequencies are preferable calculated and/or the consensus matrix is preferably prepared based on a random selection of TIS from the species of interest. Even more preferably, the relative frequencies are calculated and/or the consensus matrix is prepared based on in principle all TIS available from the species of interest.

In the event that insufficient TIS are available for the species of interest, the consensus matrix may involve use of TIS from closely related species. Thus, all or only a proportion of the TIS analysed may be from a closely related species.

For example, the gene information file (gff3) for any species of interest may be used to identify the start codon of the coding sequence (CDS) of each gene. The sequence −10 bp to +13 bp, preferably −6 bp to +9 bp surrounding the ATG (AUG) start codon, where A denotes the position “+1” may be considered as TIS. The TIS for each gene may be extracted from genome sequence of respective species using suitable software, such as BEDtools getfasta command, e.g. as described in Ref. 11. Genomic sequence information may be retrieved in any manner. For example, databases comprising genomic sequences of many different species are publicly available and include for example the nucleotide database provided by NCBI, GenBank, GrainGenes, Chenopodium, InterOmics, BnPIR or Ensembl Genome.

Examples of consensus matrices of rice, barley and fungus (Aspergillus oryzae, Koji mold):

These consensus matrices were prepared using sequences from the following databases:
Rice: Oryza sativa Japonica Group (assembly IRGSP-1.0)
Barley: The third version of the reference genome sequence assembly of barley cv. Morex [Morex V3]
Fungus: Aspergillus oryzae RIB40 (assembly ASM18445v3)

In some embodiments of the current invention, the organism is barley and the consensus matrix may preferably be the following. The consensus matrix shown in Table 2 has been prepared by analysing 19,183 genes in the barley genome and the consensus initiation sequence surrounding ATG (AUG) was elucidated. The inventions demonstrates that exchanging single nucleotides in proximity to ATG lead to altered translation initiation efficiency (see Examples 1 and 2 below).

TABLE 2
Consensus matrix obtained for barley H. vulgare based
on 19183 sequences. Beneath each nucleotide, the relative
frequency of said nucleotide is indicated in percentage.

| H. vulgare | |
|---|---|
| 19183 seq. | Nucleotide |
| Position | Relative frequency of the nucleotide |

| | | | | |
|---|---|---|---|---|
| −6 | G | C | A | T |
| | 33.6 | 24.8 | 22.6 | 19.1 |
| −5 | C | G | A | T |
| | 36.2 | 22.7 | 21.0 | 20.0 |
| −4 | G | C | A | T |
| | 27.8 | 27.7 | 27.4 | 17.2 |
| −3 | G | A | C | T |
| | 42.4 | 26.3 | 18.6 | 12.7 |
| −2 | C | A | G | T |
| | 44.2 | 27.2 | 16.3 | 12.3 |
| −1 | C | G | A | T |
| | 38.2 | 34.3 | 19.0 | 8.5 |
| 4 | G | C | A | T |
| | 55.3 | 16.5 | 16.1 | 12.0 |
| 5 | C | A | G | T |
| | 43.5 | 24.8 | 17.0 | 14.6 |
| 6 | G | C | T | A |
| | 42.0 | 24.5 | 19.1 | 14.4 |
| 7 | G | A | C | T |
| | 33.8 | 25.7 | 22.4 | 18.2 |
| 8 | C | T | A | G |
| | 33.8 | 22.4 | 21.9 | 21.8 |
| 9 | C | G | T | A |
| | 37.9 | 34.7 | 14.3 | 13.1 |

In some embodiments of the current invention, the organism is rice and the consensus matrix may preferably be the following:

TABLE 3
Consensus matrix obtained for rice O. sativa subsp. Japonica
based on 20637 sequences. Beneath each nucleotide, the relative
frequency of said nucleotide is indicated in percentage.

| O. sativa subsp. | |
|---|---|
| japonica 20637 seq. | Nucleotide |
| Position | Relative frequency of the nucleotide |

| | | | | |
|---|---|---|---|---|
| −6 | G | C | A | T |
| | 35.2 | 23.6 | 20.7 | 20.6 |
| −5 | C | G | T | A |
| | 35.8 | 22.8 | 21.1 | 20.3 |
| −4 | G | C | A | T |
| | 29.7 | 27.5 | 27.1 | 15.7 |
| −3 | G | A | C | T |
| | 43.1 | 25.6 | 16.5 | 14.8 |
| −2 | C | A | G | T |
| | 44.7 | 26.2 | 16.7 | 12.4 |
| −1 | C | G | A | T |
| | 37.5 | 33.3 | 20.3 | 8.9 |
| 4 | G | A | C | T |
| | 58.7 | 15.6 | 14.6 | 11.2 |
| 5 | C | A | G | T |
| | 43.1 | 25.3 | 17.4 | 14.1 |
| 6 | G | C | T | A |
| | 44.3 | 23.1 | 18.3 | 14.3 |
| 7 | G | A | C | T |
| | 35.0 | 25.7 | 21.1 | 18.1 |
| 8 | C | G | A | T |
| | 36.3 | 22.0 | 21.9 | 19.7 |
| 9 | G | C | T | A |
| | 37.5 | 32.7 | 15.7 | 14.1 |

For oat, quinoa, durum wheat and Brassica napus the following databases may be used for sequence information:
Oat: Assembly and annotations of Avena sativa—OT3098 v1, PepsiCo.
Quinoa: Chenopodium quinoa genome sequence (accession P1614886) at ChenopodiumDB.
Durum wheat: The Svevo cultivar genome.
Brassica napus: The Brassica napus pan-genome information resource (BnPIR).

In some embodiments of the current invention, the organism is Aspergillus oryzae and the consensus matrix may preferably be the following. The consensus matrix shown in Table 4 has been prepared by analyzing 7,442 genes in the Aspergillus oryzae genome and the consensus initiation sequence surrounding ATG (AUG) was elucidated.

TABLE 4
Consensus matrix obtained for Aspergillus oryzae based
on 7442 sequences. Beneath each nucleotide, the relative
frequency of said nucleotide is indicated in percentage.

| Aspergillus oryzae | |
|---|---|
| 7442 seq. | Nucleotide |
| Position | Relative frequency of the Nucleotide |

| | | | | |
|---|---|---|---|---|
| −6 | G | A | T | C |
| | 26.0 | 25.9 | 25.8 | 22.3 |
| −5 | C | T | A | G |
| | 31.9 | 30.2 | 24.2 | 13.7 |
| −4 | C | A | T | G |
| | 47.5 | 25.2 | 14.5 | 12.8 |
| −3 | A | G | C | T |
| | 61.5 | 22.1 | 8.6 | 7.7 |
| −2 | A | C | G | T |
| | 36.3 | 33.0 | 10.7 | 19.9 |
| −1 | C | A | G | T |
| | 35.9 | 31.0 | 18.8 | 14.3 |
| 4 | G | A | T | C |
| | 37.7 | 22.3 | 20.1 | 19.8 |
| 5 | C | A | T | G |
| | 46.4 | 23.5 | 15.3 | 14.8 |
| 6 | T | C | G | A |
| | 30.2 | 27.3 | 24.0 | 18.6 |
| 7 | G | A | C | T |
| | 27.4 | 25.4 | 24.9 | 22.3 |
| 8 | C | A | T | G |
| | 35.9 | 25.8 | 22.3 | 16.0 |
| 9 | C | T | A | G |
| | 36.5 | 23.6 | 21.1 | 18.8 |

### Consensus Sequence and Substitutions of the TIS:

Whereas the consensus matrix indicates the relative frequencies of each nucleotide at each position, it may also be useful to prepare a consensus sequence for each species. The consensus sequence comprises the sequence of the TIS containing the nucleotide with the highest relative frequency at each position. If more than one nucleotide has equally high relative frequency, e.g. a relative frequency of +/−1 percentage point, they may all be considered as having the “highest relative frequency”. In such cases, the consensus sequence may comprise more than one nucleotide on a given position.

For example, in some embodiments, the organism is barley and the consensus sequence comprises the nucleotide sequence from 5′ to 3′ GCGGCCATGGCGGCC (SEQ ID NO: 1).

In other embodiments, the organism is the fungus Aspergillus oryzae and the consensus sequence comprises the nucleotide sequence from 5′ to 3′ GCCAACATGGCTGCC (SEQ ID NO: 6).

In preferred embodiments, the mutation is a single nucleotide substitution.

In preferred embodiments of the methods or the organisms of the present invention, the one or more mutation(s) is/are compared to the parent organism, for instance the parent organism from which a variant containing said one or more mutation(s) is generated or isolated. In further embodiments, the one or more mutation(s) are compared to a wild-type organism, for instance this can be the case in which a wild type organism is used as parent organism from which the variant organism is generated or isolated according to the methods of the present invention.

In some embodiments, the generated or isolated variant organism carries substitution(s) of one or more nucleotide(s) in the TIS of the endogenous gene, wherein the substitution(s) to a nucleotide(s) identified as having a higher relative frequency in the consensus matrix of the species at that position. In particular, the substitution may be a substitution(s) to a nucleotide(s) having at least 2% points, such as at least 5% points, for example at least 10% points, such as at least 15% points, for example at least 20% points, for example at least 25% points, such as at least 30% points, for instance at least 35% points, such as at least 38% points, for instance at least 40% points, such as in the range of 2 to 40% points, for example in the range of 5 to 40% points higher relative frequency, thereby increasing the probability of higher expression levels of the protein of interest.

In other embodiment, the substitution may be a substitution(s) to a nucleotide(s) identified as having a lower relative frequency in the consensus matrix of the species at that position. In particular, the substitution may be a substitution(s) to a nucleotide(s) having at least 2% points, such as at least 5% points, for example at least 10% points, such as at least 13% points, for example at least 15% points, for example in the range of 2 to 40% points, such as in the range of 5 to 40% points lower relative frequency, thereby increasing the probability of lower expression levels of the protein of interest.

The person skilled in the art will appreciate that in some cases the TIS of the endogenous gene of interest in the parent organism may already contain the nucleotides identified as having the highest (or lowest) relative frequency in the consensus matrix at all nucleotide positions, thus not allowing for increasing the probability of higher (respectively lower) expression levels of the protein of interest by identifying the substitutions as described in the methods of the present invention in a variant organism. In other cases the TIS of the endogenous gene of interest may even already include the consensus sequence of the organisms of interest.

Thus, in embodiments of the invention relating to increasing the probability of higher expression, it is preferred that the gene of interest comprises a TIS, which is different to the consensus sequence of the species of interest. It is preferred that that the gene of interest comprises a TIS, which differs from the consensus sequence on at least 1, such as at least 2, for example at least 3 positions.

Similarly, in embodiments of the invention relating to increasing the probability of lower expression, it is preferred that the gene of interest comprises a TIS, which is different to the least frequent TIS (i.e. a sequence composed of the succession of nucleotides each being the least relatively frequent at each nucleotide position of the TIS in the species of interest). It is preferred that that the gene of interest comprises a TIS, which differs from the least frequent TIS on at least 1, such as at least 2, for example at least 3 positions.

As explained herein, the invention may be useful for modulating expression of in principle any gene. If the species of interest for examples is a crop, the methods may be used to increase the levels of proteins contributing to increased yield, e.g. to increase levels of starch contents and associated functional properties. Non limiting examples of genes encoding proteins the levels of which may be interesting to upregulate include starch synthase IIa (ssIIa), Morell et al. 2003 (Ref. 5)), control of grain-filling (GIF1 (GRAIN INCOMPLETE FILLING 1), Wang et al. 2008 (Ref. 6)) and grain yield (barley sucrose transporter HvSUT1, Saalbach et al. 2014, (Ref. 7)).

In some embodiments, the organism is barley, the protein of interest is barley sucrose transporter 2 HvSUT2 (A sequence of the barley SUT2 gene is provided as SEQ ID NO: 13. The coding sequence is also available as Horvu_PLANET_5H01G000900 (454678 to 459084 at the minus strand). The consensus sequence of barley comprises the nucleotide sequence from 5′ to 3′ GCGGCCATGGCGGCC (SEQ ID NO: 1) and the mutation consists in a C to G substitution of the nucleotide at position +4 resulting in a TIS sequence of TGTTCGATGGCGCCG (SEQ ID NO: 2). The resulting variant may be referred to as a “+4 (C/G) variant”, also referred to as LIM-14 as in Example 2 below.

In further embodiments, the organism is barley, the protein of interest is barley sucrose transporter 2 (HvSUT2) of SEQ ID NO: 16, and the TIS of HvSUT2 gene in said variant comprises or consists of SEQ ID NO: 2.

In some embodiments, the organism is barley, the protein of interest is beta-glucanase (A sequence of barley beta glucanase gene is provided as SEQ ID NO: 14. The coding sequence is also available as Horvu_PLANET_7H01G705600 (627422694 to 62/742,6679 at the minus strand), the consensus sequence comprises the nucleotide sequence from 5′ to 3′ GCGGCCATGGCGGCC (SEQ ID NO: 1) and the mutation consists in a C to T substitution of the nucleotide at position −3 resulting in a TIS sequence of GACTCAATGGCGAGC (SEQ ID NO: 3). The resulting variant may be referred to as a “−3 (C/T) variant”, also referred to as LIM-19 as in Example 1 below.

In further embodiment the organism is barley, the protein of interest is beta-glucanase of SEQ ID NO: 17, and the TIS of the gene encoding beta-glucanase in said variant comprises or consists of SEQ ID NO: 3.

In some embodiments, the organism is the fungus Aspergillus oryzae, the protein of interest is citrate synthase (coding sequence available as A0090102000627; Chromosome: 4; 2835276 to 2837167, NCBI GeneID 5994819, or as SEQ ID NO: 12), the consensus sequence of Aspergillus oryzae comprises the nucleotide sequence from 5′ to 3′ GCCAACATGGCTGCC (SEQ ID NO: 6) and the mutation consists in a G to A substitution of the nucleotide at position +4 resulting in a TIS sequence of TTCGACATGACTTCT (SEQ ID NO: 7). The resulting variant may be referred to as a “+4 (G/A) variant”, also referred to as CS-Lo as in Example 3 below.

In further embodiments, the organism is Aspergillus oryzae, the protein of interest is citrate synthase (CS) of SEQ ID NO: 15, and the TIS of the CS gene in said variant comprises or consists of SEQ ID NO: 7.

In some embodiments it is preferred that the substitutions are positioned in nucleotide positions upstream of the AUG or ATG codon.

In some embodiments, the substitution(s) is (are) located between positions −10 and +13.

In some embodiments, the substitution(s) is (are) located between positions −6 and −1.

In some embodiments, the substitution(s) is (are) positioned within nucleotide positions −6 to +9.

Substitution of nucleotides at certain positions of the TIS may in general lead to a stronger effect depending on the organism or species.

In some embodiments, the organism is a dicot and said substitution(s) is (are) positioned between position −6 and +9.

In some embodiments, the organism is a dicot and said substitution(s) is (are) located at (a) position(s) selected from the group consisting of +4, −3, −1, +5, −2, −4 and +6.

In some embodiments, the organism is a monocot and said substitution(s) is (are) located between position −6 and +9.

In some embodiments, the organism is a monocot and said substitution(s) is (are) located at (a) position(s) selected from the group consisting of −3, +4, +1, −1, and −2. E.g. the substitution may be located at position −3 and/or +4.

mRNA and Protein Levels Quantification

The methods of the invention are useful for modulating the levels of a protein of interest. Levels of a given protein may be determined either directly or indirectly as described in this section. Thus, the levels may be determined by determining the actual level of protein or the relative level of protein compared to a reference. It is however also comprised within the invention that the level of protein is determined indirectly, for example by determining an effect of the protein.

In some embodiments, the methods according to the invention further comprises a step of quantifying the protein of interest levels of the generated or isolated variant organism and comparing it to the parent organism either directly or indirectly.

Since the methods of the invention mainly affect the translation efficacy, it may also be of interest to determine the levels of protein of interest compared to the levels of mRNA transcript encoding said protein.

Thus, in some embodiments, the methods according to the invention further comprises step d) quantifying the level of mRNA transcript encoding the protein of interest and/or protein of interest levels of the generated or isolated variant organism and comparing it to a reference, e.g. the parent organism.

In some embodiments, the step of quantifying the levels of protein of interest levels of the generated or isolated variant organism is performed indirectly, e.g. by measuring protein activity.

The skilled person will appreciate that the relevant indirect protein activity assays depend on the protein of interest and may be for instance indirect assays based on the structure or function of the protein of interest.

In one embodiment, the protein of interest has an enzymatic activity and the step of quantifying the protein of interest levels in the generated or isolated variant organism is performed using an enzymatic activity assay for the protein of interest.

The skilled person will appreciate that enzymatic activity assays for instance include fluorescence-based assays, or colorimetric, absorbance-based assays. The assay may be performed using commercially available kits, such as using spectrophotometry-based measurements, for instance by incubating the protein of interest with a substrate and measuring the formation of a colored product, e.g. Beta-glucanase activity can be measured using purified barley beta glucan, chemically dyed (absorbance at 517 nm) and cross-linked (CPH0003, Glycospot, Denmark) using Glycospot protocol, as described in Example 1 below. Similarly, α-Amylase activity can be measured using standard methods, i.e. using the Ceralpha kit from Megazyme as described in Example 1 below.

If the protein of interest is a transcription factor, the level of transcript from a promoter regulated by said transcription factor may be determined in order to determine activity of said protein of interest, and thereby indirectly determine the levels.

In some embodiments, the protein levels are determined directly. Such methods may include use of antibodies or other binding agents specifically binding the protein of interest or one of its binding partners. Such methods could for example be ELISA based methods.

In some embodiments, the method according to the first aspect of the invention, further comprises a step of analyzing the translational efficiency of the protein of interest of the generated or isolated variant organism and comparing it to a reference, e.g. the parent organism. The translational efficiency may be calculated as the ratio of the protein level over the mRNA transcript levels.

The skilled person will appreciate that mRNA transcript levels may be measured using standard techniques. For instance the RNA may be extracted from the sample, such as a sample of the organism where the gene coding for the protein of interest is expected to be expressed, using standard RNA extraction kits e.g. the Aurum total RNA mini kit (Biorad). cDNA may then be synthetized using standard approaches e.g. iScript select (oligodT) synthesis kit (BioRad), as described in Example 2 below.

The cDNA levels may be quantified, e.g. by dPCR or qPCR analysis.

In some embodiments, the mRNA transcript level is quantified using quantitative PCR (qPCR).

Protein levels may be determined as described hereinabove or further below.

In some embodiments the protein level is quantified using targeted proteomics. The person skilled in the art will appreciate that targeted proteomics techniques may include array-based techniques or mass-spectrometry-based techniques. Such techniques may be performed quantitatively or semi-quantitatively. For example the targeted proteomics quantification may be performed using a label-free approach or labelling of peptides or proteins of interest, for instance using SRM (selected reaction monitoring).

In some embodiments, the protein of interest level is increased or decreased by at least 2.5%, preferably by at least 5%, more preferably by at least 7.5% compared to the reference, e.g. the parent organism. For instance the beta-glucanase levels were decreased by 8.6% in Example 1 and the sucrose-transporter 2 protein level increased by 6,5% in Example 2.

In some embodiments, the translational efficiency of the protein of interest of the generated or isolated variant organism is increased by at least 2.5%, preferably by at least 5%, more preferably by at least 7.5%, even more preferably by at least 10% compared to the reference, e.g. the parent organism. For instance the sucrose-transporter 2 translational efficiency was increased by 18.6% in Example 2.

In some embodiments, the translational efficiency of the protein of interest of the generated or isolated variant organism is decreased by at least 2.5%, preferably by at least 5%, more preferably by at least 7.5%, even more preferably by at least 10% compared to the reference, e.g. parent organism.

Said reference may preferably be the parent organism. However, it is also comprised within the invention that the reference is any wild type organism of the same species. Alternatively, the reference may be an organism, which is essentially identical to the variant organism except for said mutation(s) of interest.

### Phenotypic Traits

In some embodiments, the generated or isolated variant organism is a plant and the substitutions in the gene of interest encoding the protein of interest of the plant are associated with phenotypic traits and the phenotypic traits are conserved over several growing seasons, preferably over 2 growing seasons, most preferably over 3 growing seasons.

In some embodiments, the generated or isolated variant organism is barley, and the substitution is a +4 C/G substitution in the barley SUT2, (A barley SUT2 gene is provided as SEQ ID NO: 13, or is available under the accession number Horvu_PLANET_5H01G000900 (454678 to 459084 at the minus strand)), wherein the phenotypic trait is the reduction of the proportion of lighter grains (15 mg to 35 mg) and an increase of the proportion of heavier grains (35 mg to 65 mg) compared to a wild-type barley, preferably compared to the parent organism.

Sequence listing

| | |
|---|---|
| SEQ ID NO: 1 | Barley consensus sequence |
| SEQ ID NO: 2 | TIS sequence of the barley sucrose transporter 2, |
| | LIM-14 mutant |
| SEQ ID NO: 3 | TIS sequence of the barley beta glucanase, LIM-19 |
| | mutant |
| SEQ ID NO: 4 | TIS sequence of the barley beta glucanase, wild-type |
| SEQ ID NO: 5 | TIS sequence of the barley sucrose transporter 2, wild |
| | type |
| SEQ ID NO: 6 | Aspergillus oryzae consensus sequence. |
| SEQ ID NO: 7 | TIS sequence of the Aspergillus oryzae CS-Lo mutant |
| SEQ ID NO: 8 | Sequence of reference-specific detection probe labelled |
| | with hexachlorofluorescein (HEX) |
| | AO090102000627_+4 (G/A)_HEX |
| SEQ ID NO: 9 | Sequence of mutant-specific detection probe labelled |
| | with 6-carboxyfluorescein (FAM) |
| | AO090102000627_+4 (G/A)_FAM |
| SEQ ID NO: 10 | Sequence of the target specific forward primer |
| | AO090102000627_+4 (G/A)_F |
| SEQ ID NO: 11 | Sequence of the target specific reverse primer |
| | AO090102000627_+4 (G/A)_R |
| SEQ ID NO: 12 | Coding sequence of the Citrate synthase gene of |
| | Aspergillus oryzae (AO090102000627) |
| SEQ ID NO: 13 | Sequence of barley Sucrose Transporter 2 (SUT2) gene |
| SEQ ID NO: 14 | Sequence of the barley beta glucanase gene |
| SEQ ID NO: 15 | Polypeptide sequence of the Citrate synthase of |
| | Aspergillus oryzae |
| SEQ ID NO: 16 | Polypeptide sequence of the barley Sucrose |
| | Transporter 2 (SUT2) |
| SEQ ID NO: 17 | Polypeptide sequence of the barley beta glucanase |
| SEQ ID NO: 18 | TIS sequence of the Aspergillus oryzae CS, wild type |

### EXAMPLES

### Example 1: Down-Regulation of Grain Specific β-Glucanase

### Aim:

The inventors aimed at down-regulating the expression of grain-specific β-glucanase in malted barley using the approach described in the present invention

### Material and Methods

### Beta-Glucanase Activity in Malted Barley.

First the consensus matrix of the organism of interest for this example, barley, was generated (Table 2) as described in the “Consensus matrix” section above. The consensus matrix was prepared based on 19183 gene sequences and is provided in Table 2 above.

In order to increase the probability of lower expression levels of the beta-glucanase protein (A sequence of barley beta glucanase gene is provided as SEQ ID NO: 14), the TIS of the beta glucanase gene was compared to the nucleotide relative frequencies of the barley consensus matrix to identify a possible substitution to a nucleotide having a lower relative frequency at a specific position in the consensus matrix.

Based on the comparison it was decided to prepare a barley variant having a nucleotide substitution for the nucleotide with the lowest relative frequency at position −3 (T). The TIS of the gene encoding beta-glucanase of such a barley variant is provided herein as SEQ ID NO: 3.

A barley plant carrying said specific mutation was identified using the methods described in international patent application WO 2018/001884.

In brief, a library of barley grains of variety Planet were subjected to random mutagenesis and grown to maturity on the field. During harvest, grains of generation M1 were divided into sub-pools, such that all grains harvested from plants of one field plot containing approx. 300 plants were placed into the same sub-pool. gDNA was isolated from a random fraction of each sub-pool consisting of approx. 25% of the grains of each sub-pool. The method is described in more detail in international patent application WO 2018/001884 in WS1 and WS2 on p. 66-69 as well as in Examples 1 to 2 (hereby incorporated by reference).

A sub-pool containing a barley variant comprising the −3 C/T mutation of the TIS was selected using ddPCR. Subsequently, the individual barley plant carrying the −3 C/T mutation was identified from said sub-pool. More specifically, said barley variant was identified and selected as described in international patent application WO 2018/001884 in WS3 and WS4 on p. 67-72 as well as in Examples 3 to 15 using the unique assay ID BioRad: dMDS119276568 comprising primers and probes designed to identify said mutation.

The beta-glucanase (A barley beta glucanase gene sequence is provided as SEQ ID NO: 14) −3 C/T barley variant was crossed to CB-Score variant generating homozygote WT and homozygote variant plants referred to as LIM-19, or “Ho” herein. Sequencing confirmed that the gene encoding beta-glucanase of the LIM-19 barley plants contains a TIS of the sequence GACTCAATGGCGAGC (SEQ ID NO: 3).

Ten grains from two of each of these plants were germinated on wetted filter paper for 2 days in triplicates in 8 cm petri dishes. Germinated grains were snap frozen in liquid nitrogen and lyophilized before grinded for 6 minutes on a SPEX SamplePrep 2010 Geno/Grinder at maximum rpm. Proteins were extracted in 200 mM sodium acetate buffer (pH 4,5) including protease inhibitor cocktail tablet (cOmplete, Pierce), as described by manufacturer. Beta-glucanase activity was measured using purified barley beta glucan, chemically dyed (absorbance at 517 nm) and cross-linked (CPH0003, Glycospot, Denmark) by incubating for 1 hours, following instructions from Glycospot.

### Results

TABLE 5
Proteins were extracted from 2 day germinated barley grains
and 200 ul was used for the beta-glucanase enzyme assay following
manufactures instructions (Glycospot). Absorbance at 517 nm
was measured with a Tekan Spark ELISA plate reader. An average
absorbance for each genotype (WT wild-type, and Ho, LIM-19)
was calculated as well as standard error of mean (SE).

| | Absorbance | | Absorbance |
|---|---|---|---|
| Genotype | 517 nm | Genotype | 517 nm |
| WT cross #1 | 0.85 | Ho cross #3 | 0.82 |
| WT cross #1 | 0.97 | Ho cross #3 | 0.80 |
| WT cross #1 | 0.88 | Ho cross #3 | 0.77 |
| WT cross #2 | 0.91 | Ho cross #4 | 0.86 |
| WT cross #2 | 0.95 | Ho cross #4 | 0.86 |
| WT cross #2 | 0.90 | Ho cross #4 | 0.88 |
| | Avg. | | Avg. |
| | 0.91 | | 0.83 |
| | SE | | SE |
| | 0.018 | | 0.017 |

ANOVA analysis showed p=0.01 and the difference was 8,6%.

### Conclusion

Down-regulation of grain-specific β-glucanase could be achieved in malted barley, with 8.6% reduction in enzymatic activity, applying the method described in the present invention.

### Example 2: Up-Regulation of Sucrose Transporter 2 in Barley

### Aim:

The inventors aimed at up-regulating the expression of the sucrose transporter 2 protein (SUT2), associated with grain endosperm filling (Ref. 16) in barley leaf material using the approach described in the present invention.

### Material and Methods

### Sucrose Transporter 2+4 C/G Variant Kernel Weight Analysis

A variant of Sucrose transporter 2 (SUT2) protein (A barley SUT2 gene sequence is provided as SEQ ID NO: 13) at nucleotide position +4 (ATG(C/G)) was identified. First the consensus matrix of the organism of interest for this example, barley, was generated (Table 2) as described in the “Consensus matrix” section above. The consensus matrix was prepared based on 19183 gene sequences and is provided in Table 2 above.

In order to increase the probability of higher expression levels of the Sucrose transporter 2 protein (A barley Sucrose transporter 2 protein gene sequence is provided as SEQ ID NO: 13), the TIS of the Sucrose transporter 2 gene was compared to the nucleotide relative frequencies of the barley consensus matrix to identify a possible substitution to a nucleotide having a higher relative frequency at a specific position in the consensus matrix. Based on the comparison it was decided to prepare a barley variant having a nucleotide substitution for the nucleotide with the highest relative frequency at position +4 (G). The TIS of the gene encoding SUT2 of such a barley variant is provided herein as SEQ ID NO: 2.

A barley plant carrying said specific mutation was identified using the methods described in international patent application WO 2018/001884.

In brief, a library of barley grains of variety Planet were subjected to random mutagenesis and grown to maturity on the field. During harvest, grains of generation M1 were divided into sub-pools, such that all grains harvested from plants of one field plot containing approx. 300 plants were placed into the same sub-pool. gDNA was isolated from a random fraction of each sub-pool consisting of approx. 25% of the grains of each sub-pool. The method is described in more detail in international patent application WO 2018/001884 in WS1 and WS2 on p. 66-69 as well as in Examples 1 to 2 (hereby incorporated by reference).

A sub-pool containing a barley variant comprising the +4 C/G mutation of the TIS was selected using ddPCR. Subsequently, the individual barley plant carrying the +4 C/G mutation was identified from said sub-pool. More specifically, said barley variant was identified and selected as described in international patent application WO 2018/001884 in WS3 and WS4 on p. 67-72 as well as in Examples 3 to 15 using the unique assay ID BioRad: dMDS456124642 comprising primers and probes designed to identify said mutation.

The Sucrose transporter 2 (SUT2) protein (A barley SUT2 gene sequence is provided as SEQ ID NO: 13)+4 C/G barley variant was crossed to generate homozygote WT and homozygote variant plants. Sequencing confirmed that the gene encoding SUT2 of barley variant plants contains a TIS of the sequence TGTTCGATGGCGCCG (SEQ ID NO: 2).

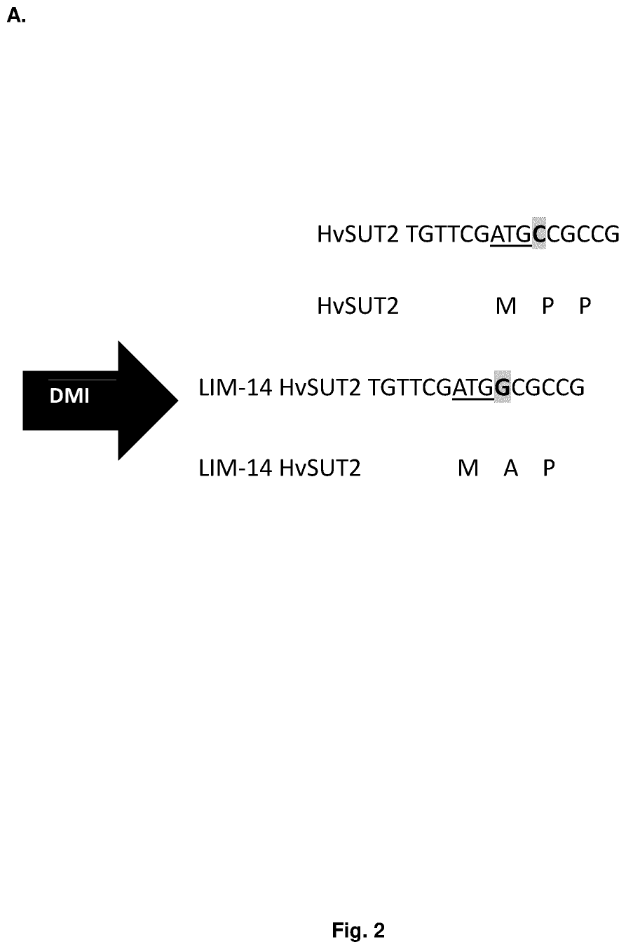

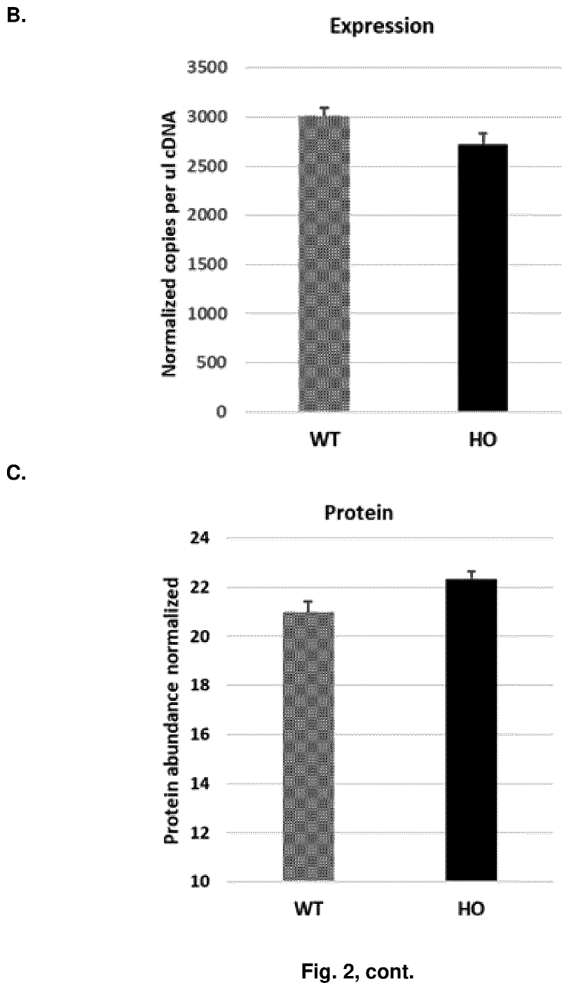

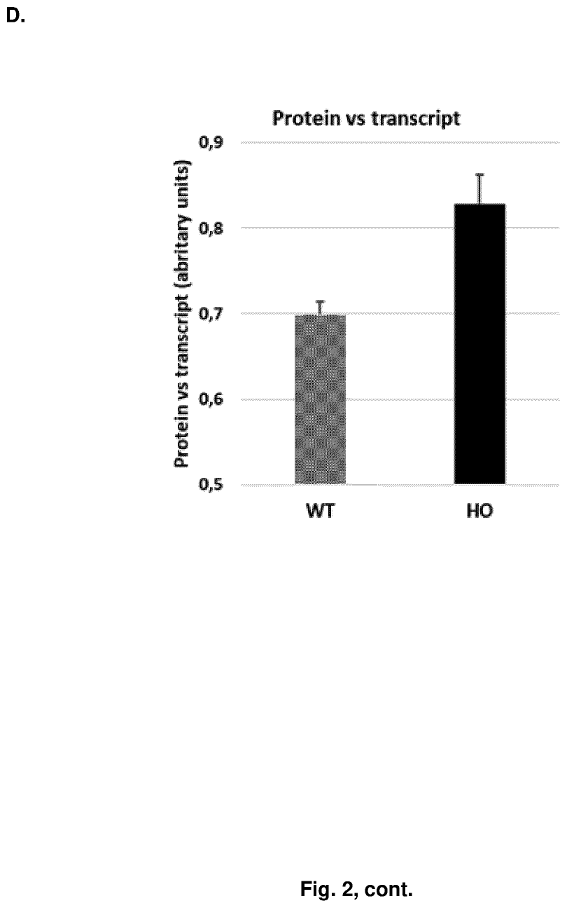

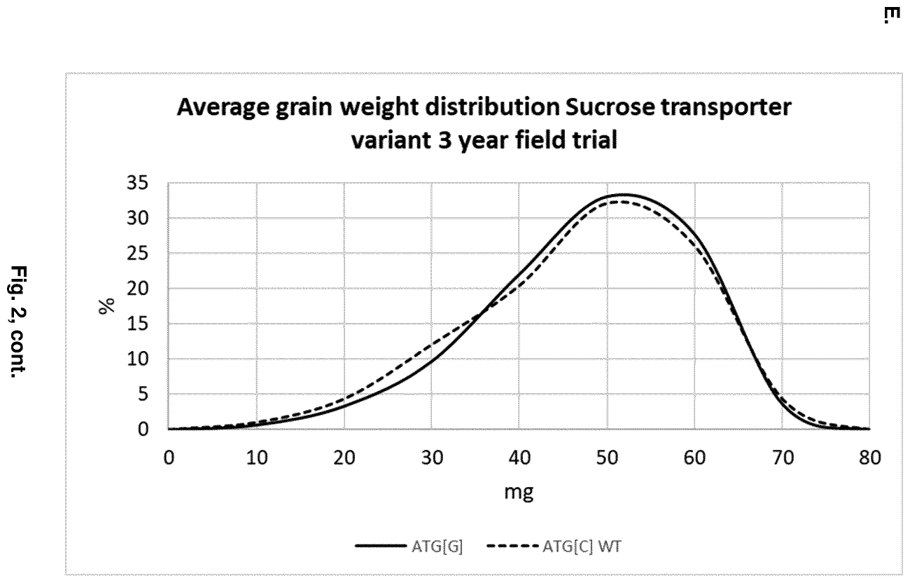

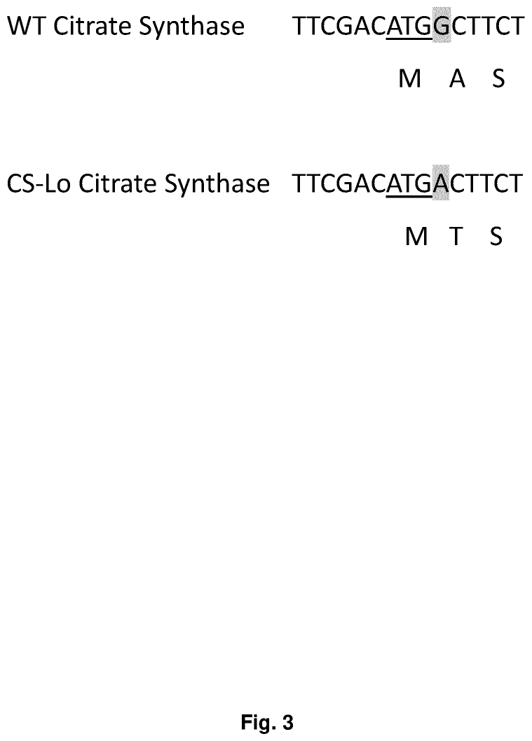

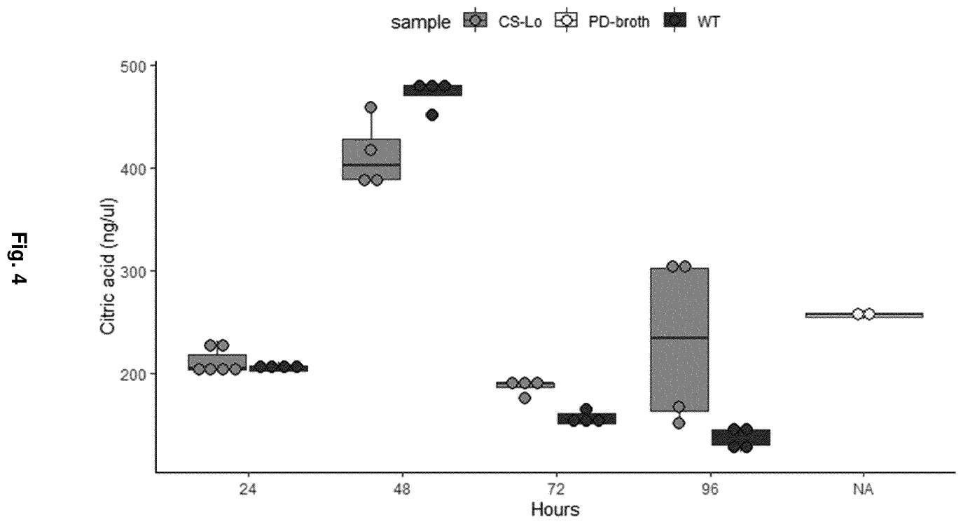

Homozygote wild type as well as homozygote variant was grown at locations in Denmark and New Zealand in 3 consecutive growing seasons and representative grains from each year were weighted and an average grain weight distribution was calculated (FIG. 2. D))

### Quantitative Gene Transcript Abundance and Targeted Proteomics Analysis

Grains from homozygote WT and homozygote variant from 8 different plots (Denmark 2020 and New Zeeland 2020) were germinated in individual appropriately watered vermiculite trays for 7 days. Approximately 100 seedlings were harvested and directly frozen in liquid nitrogen. The frozen material was grinded using a porcelain mortar and a pestle mixing grinder, and divided into two sub-samples, one for transcript abundance analysis and one for targeted proteomics analysis, which was then lyophilized. Quantitative gene expression analysis was carried out as described in (Ref. 12) using GAPDH and Pyruvate kinase as reference genes. RNA was extracted with Aurum total RNA mini kit (BioRad) and cDNA synthesized from 200 ng total RNA with iScript select (oligodT) synthesis kit (BioRad) according to manufacturer's instruction. Targeted proteomics analysis was performed by Proteomics core facility, Technical University of Denmark, Lyngby Denmark (Ref. 13 and Ref. 14). The results can be found in Table 6.

TABLE 6
Quantitative transcript abundance and protein content of native (WT) and variant
(HO) in 7 days old seedlings. An average (Avg) transcript abundance (Normalized
copies “Norm. copies per μl cDNA”), protein content (Normalized Sucrose
transporter 2 protein “Norm. HvSUT2 protein”) and protein vs. transcript
abundance for each genotype was calculated as well as standard error of mean (SE).

| | | | Ratio | | | | Ratio |
|---|---|---|---|---|---|---|---|
| | Norm. | | protein | | Norm. | | protein |
| | copies | Norm. | vs | | copies | Norm. | vs. |
| | pr μl | HvSUT2 | transcript | | pr μl | HvSUT2 | transcript |
| Genotype | cDNA | protein | * 100 | Genotype | cDNA | protein | * 100 |
| WT plot 1, | 2887 | 20.1 | 0.7 | HO plot 1, | 2847 | 22.2 | 0.8 |
| 2020 NZ | | | | 2020 NZ | | | |
| WT plot 2, | 3237 | 20.1 | 0.6 | HC plot 2, | nd | nd | nd |
| 2020 NZ | | | | 2020 NZ | | | |
| WT plot 3, | 2659 | 19.8 | 0.7 | HO plot 3, | 3066 | 21.5 | 0.7 |
| 2020 NZ | | | | 2020 NZ | | | |
| WT plot 4, | nd | nd | nd | HO plot 4, | 2172 | 21.5 | 1.0 |
| 2020 NZ | | | | 2020 NZ | | | |
| WT plot 1, | 2831 | 20.6 | 0.7 | HO plot 1, | 2759 | 23.6 | 0.9 |
| 2020 DK | | | | 2020 DK | | | |
| WT plot 2, | 3095 | 21.5 | 0.7 | HO plot 2, | 2746 | 22.0 | 0.8 |
| 2020 DK | | | | 2020 DK | | | |
| WT plot 3, | 3144 | 21.6 | 0.7 | HO plot 3, | 2556 | 22.5 | 0.9 |
| 2020 DK | | | | 2020 DK | | | |
| WT plot 4, | 3209 | 23.1 | 0.7 | HO plot 4, | 2915 | 23.1 | 0.8 |
| 2020 DK | | | | 2020 DK | | | |
| Avg | 3009 | 21.0 | 0.699 | Avg | 2722 | 22.3 | 0.829 |
| SE | 83 | 0.4 | 0.01 | SE | 109 | 0.3 | 0.03 |
| | | | | T. test | 0.06 | 0.02* | 0.005** |

### Results:

ANOVA analysis of the data presented in Table 6 showed similar transcript levels p=0.06 but that the protein product of the HvSUT2 gene (A barley SUT2 gene sequence is provided as SEQ ID NO: 13) was significantly higher in the variant p=0.02 (6,5% increase) as well as the protein vs transcript ratio p=0,005 (18.6% increase).

For each year, an average of 490 grains were individually weighted for each genotype and the distribution calculated and averaged across all years. FIG. 2.E. shows that for the three years, the variant had a reduced proportion of lighter grains (15 mg to 35 mg) and a larger proportion of heavier grains (35 mg to 65 mg) compared to the WT. The distribution was binned at 10 mg for illustration purpose.

### Conclusion

The protein levels and translational efficiency of SUT2 have been increased by 6.5% and 18,6% respectively in barley due to application of the presented technology. Further the inventors have shown that the variant with the higher translational efficiency displayed a larger proportion of heavier grains during at least 3 seasons.

### Example 3: Down-Regulation of Citrate Synthase in Aspergillus oryzae

### Aim:

The inventors aimed at down-regulating the expression of citrate synthase (CS) in Aspergillus oryzae (Koji mold) using the approach described in the present invention.

### Material and Methods

Citrate Synthase (CS) Activity in A. oryzae (Koji Mold).

First the consensus matrix of the organism of interest for this example, Aspergillus oryzae, was generated (Table 4) as described in the “Relative Frequency and Consensus matrix” section above. The consensus matrix was prepared based on 7442 gene sequences and is provided in Table 4 above.

In order to increase the probability of lower expression levels of the citrate synthase protein (encoded by the gene accessible under A0090102000627; Chromosome: 4; 2835276 to 2837167, NCBI GeneID:5994819, or by SEQ ID NO: 12), the TIS of the citrate synthase gene was compared to the nucleotide relative frequencies of the Aspergillus oryzae consensus matrix to identify a possible substitution to a nucleotide having a lower relative frequency at a specific position in the consensus matrix. Based on the comparison it was decided to prepare a Aspergillus oryzae variant having a nucleotide substitution for the nucleotide with a lower relative frequency at position +4 (A). The TIS of the gene encoding citrate synthase of said Aspergillus oryzae variant is provided herein as SEQ ID NO: 7.

### Library Construction and Mutant Identification

A mutagenized library of Aspergillus oryzae was developed using a pooling and splitting method as described in PCT/EP2017/065516. In brief, Aspergillus oryzae grown on Malt Extracted Agar (MEA) plates at 37° C. until full sporulation was observed (ca. 14 days). Spores were harvested using 0.01% Triton-X, pelleted by certification and resuspended in sterile, distilled water. Then, 8 μl MNNG were added to 1 ml of spores (ca. 7.5E+06 spores) in a 1.5 ml safe-lock reaction tube, and spores were incubated for approx. 4 hours at 37° C. and 1200 rpm on an Eppendorf Thermomixer comfort (1.5 ml), which usually resulted in a killing rate of 40%. To stop the mutagenesis, spores were pelleted by brief centrifugation and washed three times with sterile, distilled water and finally re-suspended in 1 ml sterile, distilled water. The viable spore titer was determined by plating respective dilutions on MEA plates incubated at 37° C. Subsequently, live spores were sub-pooled by plating approx. 250 spores per MEA plate in a total of 96 plates and incubated at 37° C. until full sporulation was observed (ca. 14 days).

Spores were harvested using 1 ml of 0.01% Triton-x and 50 μl were used for gDNA extraction and subsequent screening for specific mutation events. (gDNA extraction was performed by adding 30 μl NaOH 0.1 M and boiled at 95° C. for 5 min). A sub-pool containing an Aspergillus oryzae variant comprising the +4 (G/A) mutation of the TIS was selected using ddPCR. Subsequently the individual fungal spore carrying the +4 (G/A) mutation was identified from said sub-pool. Mutant identification was performed according to the ddPCR screening method described in PCT/EP2017/065516. Primers and probes were designed for the identification of the +4 (G/A) specific mutant. In order to identify the mutants, a target specific forward primer (AO090102000627_+4 (G/A)_F, SEQ ID NO: 10), a target-specific reverse primer (AO090102000627_+4 (G/A)_R, SEQ ID NO: 11), a mutant-specific detection probe labelled with 6-carboxyfluorescein (FAM) (AO090102000627_+4 (G/A) FAM, SEQ ID NO: 9)_and a reference-specific detection probe labelled with hexachlorofluorescein (HEX) (AO090102000627_+4 (G/A)_HEX, SEQ ID NO: 8) were designed.

Screening of the library of 96×250 cells (total library size 24.000 cells) resulted in the identification of the +4 (G/A) mutant.

The citrate synthase gene A0090102000627 (Chromosome: 4; 2835276 to 2837167, NCBI GeneID 5994819, or SEQ ID NO: 12)+4 (G/A) fungal spore variant was grown in a MEA plates and harvested spores referred to as CS-Lo herein.

Ten mL of Potato Dextrose (PD) broth was inoculated with a total of 10E+07 CS-Lo spores and incubated at 30° C. and 170 rpm for 24, 48, 72 and 96 hours. The same process, with the same amount of inoculation material was followed using Aspergillus oryzae wild type (WT) spores. All inoculations were done in biological duplicates (except for the CS-Lo mutant done at 24 hours in triplicate). For every time point, two cultures containing CS-Lo and two cultures containing WT spores were centrifuged at 5000 rpm for 20 min and the supernatant was removed, filtrated (using filter paper disk 15 mm diameter) and kept at −20° C. for further analysis.

Citrate synthase is the first enzyme of the TCA (tricarboxylic acid cycle) that catalyzes the condensation of oxaloacetate and acetyl-CoA to form Citric Acid (Ref. 17). According to Ghulam et al. 2014 (Ref. 18) enhanced production of Citric acid was observed in mutant strains of Aspergillus niger in which citrate synthase gene was hyper-expressed. Thus, the effect of +4 (G/A) mutation was indirectly accessed through quantification of Citric acid by high-performance liquid chromatography (HPLC) (Agilent 1100 HPLC equipped with a Prevail™ organic acid column 150×4.6 mm) at 40° C. An organic acid standard, containing citric acid with 5, 20, 40, 60 and 100 ng/μl were used to calculate calibration curve. Each sample was run in technical duplicates by 5 and 10 times dilution in phosphate buffer. The results are presented in Table 7 below.

### Results:

TABLE 7
Levels of Citric acid were estimated after 24, 48, 72, and
96 hours of fermentation in PD Broth using HPLC in two biological
replicates and two technical replicates (dilutions) per time
point. Citric acid in PD Broth was also calculated to estimate
background citric acid levels. An average Citric acid concentration
(ng/μl) for each genotype (WT wild type and CS-Lo) was
calculated as well as standard error of mean (SE). Two groups
showing statistically significance difference by T-test analysis
are marked by “*”.

| | | | | Citric | | |
|---|---|---|---|---|---|---|
| Genotype | Hours | Replicate | Dilution | acid (ng/ul) | | |
| WT | 24 | 1 | 5 | 203.7 | | |
| WT | 24 | 1 | 10 | 201.3 | | |
| WT | 24 | 2 | 5 | 206.0 | Avg. | SE |
| WT | 24 | 2 | 10 | 211.6 | 205.6 | 2.2 |
| CS-Lo | 24 | 1 | 5 | 202.5 | | |
| CS-Lo | 24 | 1 | 10 | 203.7 | | |
| CS-Lo | 24 | 2 | 5 | 204.8 | | |
| CS-Lo | 24 | 2 | 10 | 204.5 | | |
| CS-Lo | 24 | 3 | 5 | 222.2 | Avg. | SE |
| CS-Lo | 24 | 3 | 10 | 231.3 | 211.5 | 5.0 |
| WT | 48 | 1 | 5 | 481.5 | | |
| WT | 48 | 1 | 10 | 451.5 | | |
| WT | 48 | 2 | 5 | 480.3 | Avg. | SE |
| WT | 48 | 2 | 10 | 478.4 | 472.9* | 7.2 |
| CS-Lo | 48 | 1 | 5 | 417.6 | | |
| CS-Lo | 48 | 1 | 10 | 459.4 | | |
| CS-Lo | 48 | 2 | 5 | 389.2 | Avg. | SE |
| CS-Lo | 48 | 2 | 10 | 388.4 | 413.6* | 16.7 |
| WT | 72 | 1 | 5 | 159.1 | | |
| WT | 72 | 1 | 10 | 151.6 | | |
| WT | 72 | 2 | 5 | 165.0 | Avg. | SE |
| WT | 72 | 2 | 10 | 149.2 | 156.2 | 3.6 |
| CS-Lo | 72 | 1 | 10 | 190.2 | | |
| CS-Lo | 72 | 1 | 5 | 189.9 | | |
| CS-Lo | 72 | 2 | 10 | 176.0 | Avg. | SE |
| CS-Lo | 72 | 2 | 5 | 189.9 | 186.5 | 3.5 |
| WT | 96 | 1 | 5 | 132.6 | | |
| WT | 96 | 1 | 10 | 123.1 | | |
| WT | 96 | 2 | 5 | 146.8 | Avg. | SE |
| WT | 96 | 2 | 10 | 144.5 | 136.8 | 5.5 |
| CS-Lo | 96 | 1 | 5 | 305.5 | | |
| CS-Lo | 96 | 1 | 10 | 301.6 | | |
| CS-Lo | 96 | 2 | 5 | 167.7 | Avg. | SE |
| CS-Lo | 96 | 2 | 10 | 150.8 | 231.4 | 41.8 |
| PD-broth | NA | 2 | 5 | 252.6 | Avg. | SE |
| PD-broth | NA | 3 | 10 | 261.3 | 256.9 | 4.3 |

T-test analysis of the citric acid values measured between the WT and CS-LO groups at 48H showed t(4)=−3.3 and p=0.03* and the difference is 12.5%. Citric acid levels in additional timepoints were below background levels detected in PD Broth indicating no production of Citric acid by both WT and CS-Lo strain.

### Conclusion

Down-regulation of Citrate synthase could be achieved in Aspergillus oryzae, with 12.5% reduction in Citric acid production, applying the method described in the present invention.

### REFERENCES

Ref. 1. C. Merchante, A. N. Stepanova, J. M. Alonso, Translation regulation in plants: an interesting past, an exciting present and a promising future. Plant J. 90, 628-653 (2017).
Ref 2 P. Gupta, L. Rangan, T. V. Ramesh, M. Gupta, Comparative analysis of contextual bias around the translation initiation sites in plant genomes. Journal of Theoretical Biology. 404, 303-311 (2016).
Ref 3 Y. Kim, G. Lee, E. Jeon, E. ju Sohn, Y. Lee, H. Kang, D. wook Lee, D. H. Kim, I. Hwang, The immediate upstream region of the 5′-UTR from the AUG start codon has a pronounced effect on the translational efficiency in Arabidopsis thaliana. Nucleic Acids Research. 42, 485-498 (2014).
Ref 4 S. Agarwal, S. Jha, I. Sanyal, D. V. Amla, Effect of point mutations in translation initiation context on the expression of recombinant human a1-proteinase inhibitor in transgenic tomato plants. Plant Cell Rep. 28, 1791-1798 (2009).
Ref 5: M. K. Morell, B. Kosar-Hashemi, M. Cmiel, M. S. Samuel, P. Chandler, S. Rahman, A. Buleon, I. L. Batey, Z. Li, Barley sex6 mutants lack starch synthase IIa activity and contain a starch with novel properties. Plant J. 34, 173-185 (2003).
Ref 6: E. Wang, J. Wang, X. Zhu, W. Hao, L. Wang, Q. Li, L. Zhang, W. He, B. Lu, H. Lin, H. Ma, G. Zhang, Z. He, Control of rice grain-filling and yield by a gene with a potential signature of domestication. Nat Genet. 40, 1370-1374 (2008).
Ref 7: 1. Saalbach, I. Mora-Ramfrez, N. Weichert, F. Andersch, G. Guild, H. Wieser, P. Koehler, J. Stangoulis, J. Kumlehn, W. Weschke, H. Weber, Increased grain yield and micronutrient concentration in transgenic winter wheat by ectopic expression of a barley sucrose transporter. Journal of Cereal Science. 60, 75-81 (2014).
Ref 8: J. S. Gootenberg, O. O. Abudayyeh, J. W. Lee, P. Essletzbichler, A. J. Dy, J. Joung, V. Verdine, N. Donghia, N. M. Daringer, C. A. Freije, C. Myhrvold, R. P. Bhattacharyya, J. Livny, A. Regev, E. V. Koonin, D. T. Hung, P. C. Sabeti, J. J. Collins, F. Zhang, Nucleic acid detection with CRISPR-Cas13a/C2c2. Science. 356, 438-442 (2017).
Ref 9. K. A. Molla, S. Sretenovic, K. C. Bansal, Y. Qi, Precise plant genome editing using base editors and prime editors. Nat. Plants. 7, 1166-1187 (2021).
   Ref 10. S. Knudsen, T. Wendt, C. Dockter, H. C. Thomsen, M. Rasmussen, M. E. Jorgensen, Q. Lu, C. Voss, E. Murozuka, J. T. Osterberg, J. Harholt, I. Braumann, J. A. Cuesta-Seijo, S. Bodevin, L. T. Petersen, M. Carciofi, P. R. Pedas, J. O. Husum, M. T. Simmelsgaard Nielsen, K. Nielsen, M. K. Jensen, L. A. Møller, Z. Gojkovic, A. Striebeck, K. Lengeler, R. T. Fennessy, M. Katz, R. Garcia Sanchez, N. Solodovnikova, J. Forster, O. Olsen, B. L. Møller, G. B. Fincher, B. Skadhauge, “FIND-IT: Ultrafast mining of genome diversity” (preprint, Genetics, 2021), doi:10.1101/2021.05.20.444969.
Ref 11. A. R. Quinlan, I. M. Hall, BEDTools: a flexible suite of utilities for comparing genomic features. Bioinformatics. 26, 841-842 (2010).
Ref 12. The Digital MIQE Guidelines Update: Minimum Information for Publication of Quantitative Digital PCR Experiments for 2020. Clinical Chemistry. 66, 1464-1464 (2020).
Ref 13. N. Rauniyar, Parallel Reaction Monitoring: A Targeted Experiment Performed Using High Resolution and High Mass Accuracy Mass Spectrometry. IJMS. 16, 28566-28581 (2015).
Ref 14. M. van Bentum, M. Selbach, An Introduction to Advanced Targeted Acquisition Methods. Molecular & Cellular Proteomics. 20, 100165 (2021).
Ref. 15. M. R. Green, J. Sambrook, J. Sambrook, Molecular cloning: a laboratory manual (Cold Spring Harbor Laboratory Press, Cold Spring Harbor, N.Y, 4th ed., 2012).
   Ref 16. V. Radchuk, D. Riewe, M. Peukert, A. Matros, M. Strickert, R. Radchuk, D. Weier, H.-H. Steinbifß, N. Sreenivasulu, W. Weschke, H. Weber, Down-regulation of the sucrose transporters HvSUT1 and HvSUT2 affects sucrose homeostasis along its delivery path in barley grains. Journal of Experimental Botany. 68, 4595-4612 (2017).
Ref 17. S. Beeckmans, Some structural and regulatory aspects of citrate synthase. International Journal of Biochemistry. 16, 341-351 (1984).
Ref 18. G. Mustafa, A. Tahir, M. Asgher, M.-Rahman, A. Jamil, Comparative sequence analysis of citrate synthase and 18S ribosomal DNA from a wild and mutant strains of Aspergillus niger with various fungi. Bioinformation. 10, 1-7 (2014)

### Items

The invention may further be defined by anyone of the following items:
1. A method for modulating levels of a protein of interest in a eukaryotic organism of a species of interest, or a method of identifying a eukaryotic organism of a species of interest having modulated levels of a protein of interest, said method comprising the steps of:
   a) obtaining the gDNA sequence of the translation initiation sequence (TIS) of a gene of interest encoding the protein of interest of the species,
   b) comparing the gDNA sequence of the TIS of said gene with the relative frequency of each nucleotide in one or more positions of the TIS, preferably in each position of the TIS in said species or a highly similar species,
   c) generating a variant organism carrying mutation(s) or isolating a variant organism carrying mutation(s), wherein said mutation(s) is (are) substitution(s) of one or more nucleotide(s) in the TIS of the endogenous gene,
   wherein a substitution to a nucleotide identified as having a higher relative frequency at that position increases the probability of higher levels of the protein of interest, and
   wherein a substitution to a nucleotide identified as having a lower relative frequency at that position increases the probability of lower levels of the protein of interest, and with the proviso that the eukaryotic organism is not human.
2. The method according to item 1, further comprising a step d) of quantifying the protein of interest levels of the generated or isolated variant organism and comparing it to the parent organism.
3. The method according to item 1, further comprising a step d) of quantifying the mRNA transcript and/or protein of interest levels of the generated or isolated variant organism and comparing it to the parent organism.
4. The method according to any one of items 2 to 3, wherein step d) is performed using indirect protein and/or mRNA activity.
5. The method according to any one of items 2 to 4, wherein the protein of interest has an enzymatic activity and wherein step d) is performed using an enzymatic activity assay for the protein of interest.
6. The method according to any one of the preceding items, further comprising a step e) of analyzing the translational efficiency of the protein of interest of the generated or isolated variant organism and comparing it to the wild-type organism, wherein the translational efficiency is calculated as the ratio of the protein level over the mRNA transcript levels.
7. The method according to any one of items 2 to 6, wherein the protein level is quantified using targeted proteomics.
8. The method according to any one of items 3 to 7, wherein the mRNA transcript level is quantified using quantitative qPCR.
9. The method according to any one of the preceding items, wherein the protein of interest level is increased or decreased by at least 2.5%, preferably by at least 5%, more preferably by at least 7.5% compared to the parent organism.
10. The method according to any one of the preceding items, wherein the translational efficiency of the protein of interest of the generated or isolated variant organism is increased by at least 2.5%, preferably by at least 5%, more preferably by at least 7.5%, even more preferably by at least 10% compared to the parent organism.
11. The method according to any one of the preceding items, wherein the translational efficiency of the protein of interest of the generated or isolated variant organism is decreased by at least 2.5%, preferably by at least 5%, more preferably by at least 7.5%, even more preferably by at least 10% compared to the parent organism.
12. The method according to any one of the preceding items, wherein the variant organism comprising the substitution(s) is isolated using a single nucleotide polymorphism identification technology.
13. The method according to any one of the preceding items, wherein the variant is generated and/or identified using programmable nucleases.
14. The method according to any one of the preceding items, wherein the variant is generated and/or identified using a CRISPR guide RNA system.
15. The method according to any one of the preceding items, wherein the variant is generated and/or identified using a base editor.
16. The method according to any one of the preceding items, wherein the variant is generated and/or identified using a prime editor.
17. The method according to item 12, wherein the single nucleotide polymorphism identification technology comprises the steps of:
   a. providing a pool comprising a plurality of said organisms of the species of interest, or reproductive parts thereof, representing a plurality of different genotypes;
   b. dividing said pool into one or more sub-pools of organisms, or reproductive parts thereof, wherein each sub-pool comprises more than one copy of organisms of each genotype or reproductive parts thereof;
   c. obtaining at least two random fractions of said sub-pool, wherein said fractions in theory each comprises organisms representing each genotype of said sub-pool
   d. preparing gDNA samples from one fraction of each sub-pool, while maintaining the at least one fraction of each sub-pool for potential multiplication of organisms of each genotype within said sub-pool;
   e. detecting said substitution(s) in said gDNA samples, thereby identifying sub-pool(s) comprising organism(s) or reproductive parts thereof comprising said substitution(s)
   f. identifying from said identified sub-pool one or more individual organisms comprising said substitution(s).
18. The method according to item 17 wherein the step the step (e) of detecting said substitution(s) in said gDNA samples is performed by sequencing-based technology.
19. The method according to any one of items 17 to 18, wherein the step (e) of detecting said substitution(s) in said gDNA samples comprises:
   i. performing a plurality of PCR amplifications, each comprising the gDNA sample from one sub-pool, wherein each PCR amplification comprises a plurality of compartmentalised PCR amplifications, each comprising part of said gDNA sample, one or more set(s) of primers each set flanking a target sequence comprising the TIS of the gene of interest encoding the protein of interest of the species and PCR reagents, thereby amplifying the target sequence(s);
   ii. detecting PCR amplification product(s) comprising one or more target sequence(s) comprising said substitution(s), thereby identifying sub-pool(s) comprising organism(s) or reproductive parts thereof comprising said substitution(s);
20. The method according to item 19, wherein the PCR amplification(s) of step (i) is (are) performed by a method comprising the following steps:
   a. preparing one or more PCR amplifications comprising the gDNA sample, one or more set(s) of primers each set flanking a target sequence and PCR reagents;
   b. partitioning said PCR amplification(s) into a plurality of spatially separated compartments;
   c. performing PCR amplification(s);
   d. detecting PCR amplification products,
   wherein said spatially separated compartments for example are droplets, such as a water-oil emulsion droplets, wherein each droplet for example has an average volume in the range of 0.1 to 10 nL,
   and/or
   wherein each PCR for example is compartmentalised into in the range of 1000 to 100,000 spatially separated compartments.
21. The method according to any one of items 19 or 20, wherein the PCR reagents comprises:
   a. one or more mutation detection probes, wherein each mutation detection probe(s) comprise(s) an oligonucleotide optionally linked to detectable means, wherein the oligonucleotide is identical to—or complementary to—a target sequence, including a predetermined substitution of the TIS of the gene of interest; and/or
   b. one or more reference detection probe(s), wherein each reference detection probe(s) comprise(s) an oligonucleotide optionally linked to detectable means, wherein the oligonucleotide is identical to—or complementary to—a target sequence, including a reference TIS of the gene of interest;
wherein the mutant detection probe(s) optionally is (are) linked to a fluorophore and a quencher, and/or the reference detection probe optionally is linked to a different fluorophore and a quencher.
22. The method according to any one of items 17 to 21, wherein said pool of organisms comprises at least 10,000, preferably at least 100,000, yet more preferably at least 500,000 organisms, or reproductive parts thereof, with different genotypes.
23. The method according to any one of items 17 to 22, wherein said pool of organisms is generated by subjecting a plurality of organisms of reproductive parts thereof to a step of random mutagenesis.
24. The method according to item 23, wherein at least 10,000, preferably at least 100,000, yet more preferably at least 500,000 organisms, or reproductive parts thereof, are subjected to random mutagenesis.
25. The method according to any one of items 17 to 24, wherein the methods comprise a step of reproduction of the organisms, or reproductive parts thereof, within the pool, and wherein said step of reproducing may be performed simultaneously with, or subsequent to, step b) of dividing the organisms into sub-pools.
26. The method according to any one of the preceding items, wherein the organism is a plant, and wherein all seeds of a given plant are placed into the same sub-pool.
27. A eukaryotic organism comprising one or more mutation(s), wherein the mutation(s) is(are) in the translation initiation sequence (TIS) of a gene coding for a protein of interest, and wherein said mutation(s) is a(are) substitution(s) of one or more nucleotide(s) in the TIS of the endogenous gene,
   wherein a substitution to a nucleotide identified as having a higher relative frequency at that position increases the probability of higher expression levels of the protein of interest, and wherein
   a substitution to a nucleotide identified as having a lower relative frequency at that position increases the probability of lower expression levels of the protein of interest.
28. The method or the organism according to any one of the preceding items, wherein the mutation is a single nucleotide mutation.
29. The method or the organism according to any one of the preceding items, wherein the one or more mutation(s) is/are compared to the parent organism.
30. The method or the organism according to any one of the preceding items, wherein the parent organism is a wild-type organism.
31. The method or the organism according to any one of the preceding items, wherein the organism is a fungus or a plant.
32. The method or the organism according to item 31, wherein the fungus is a yeast or a filamentous fungus.
33. The method or the organism according to item 32, wherein the yeast is of the genus Saccharomyces, such as S. cerevisiae or S. pastorianus.
34. The method or the organism according to item 32, wherein the filamentous fungus is of the genera Aspergillus or Fusarium.
35. The method or the organism according to item 31, wherein the plant is selected from the group consisting of flowering plants, conifers, gymnosperms, ferns, clubmosses, hornworts, liverworts, mosses, green algae and brown algae.
36. The method or the organism according to any one of items 31 and 35, wherein the plant is selected from the group consisting of monocots or dicots.
37. The method or the organism according to item 36, wherein the monocot or dicot is selected from the group consisting of: barley, rice, wheat, corn, oat, peas, yellow peas, chickpeas, faba beans, rapeseed, durum wheat, risotto rice, bitter gourd, quinoa, alfalfa, lupin and soy.
38. The method or the organisms according to any one of the preceding items wherein the substitution(s) is (are) located between position −10 and +13.
39. The method or the organisms according to any one of the preceding items wherein the substitution(s) is (are) located between position −6 and +9.
40. The method or the organism according to any one of the preceding items, wherein the substitution(s) is (are) located between position −6 and −1.
41. The method or the organism according to any one of items 1 to 36, wherein the organisms is a dicot and said substitution(s) is (are) located between position −6 and +9.
42. The method or the organism according to any one of the preceding items, wherein the organisms is a dicot and said substitution(s) is (are) located at (a) position(s) selected from the group consisting of +4, −3, −1, +5, −2, −4 and +6.
43. The method or the organism according to any one of items 1 to 36, wherein the organisms is a monocot and said substitution(s) is (are) located between position −6 and +9.
44. The method or the organism according to any one of the preceding items, wherein the organisms is a monocot and said substitution(s) is (are) located at (a) position(s) selected from the group consisting of −3, +4, +1, −1, and −2.
45. The method or the organism according to any one of the preceding items, wherein the method comprises generating a consensus matrix of the species of interest, wherein the consensus matrix indicates the relative frequency of each nucleotide in each position of TIS in said species.
46. The method or the organism according to item 45, wherein the consensus matrix is obtained by analyzing the TIS sequence of more than 100 genes, for example more than 5000, such as more than 10000, for example more than 15000 genes, such as more than 25000 genes, for example more than 30000 genes of the organism of interest, determining the relative frequency of each nucleotide A, T, G, and C at each nucleotide position of the TIS around the ATG start codon.
47. The method according to any one of the preceding items, wherein the relative frequency of each nucleotide is obtained by analyzing the TIS sequence of more than 100 genes, for example more than 5000, such as more than 10000, for example more than 15000 genes, such as more than 25000 genes, for example more than 30000 genes of the organism of interest or a highly similar species.
48. The method according to any one of the preceding items, wherein the relative frequency of each nucleotide is obtained by analyzing the TIS sequence of more than 100 genes, for example more than 5000, such as more than 10000, for example more than 15000 genes, such as more than 25000 genes, for example more than 30000 genes of the organism of interest.
49. The method according to any one of the preceding items, wherein the relative frequencies is calculated by retrieving TIS sequences of more than 100 genes, for example more than 5000, such as more than 10000, for example more than 15000 genes, such as more than 25000 genes, for example more than 30000 genes of the organism of interest from databases of genomic sequences.
50. The method or the organism according to any one of items 45 to 49, wherein the consensus matrix has the following format

| Organism | Nucleotide | | | |
|---|---|---|---|---|
| Position | Relative frequency of the nucleotide | | | |
| −6 | Nucleotide 1 | Nucleotide 2 | Nucleotide 3 | Nucleotide 4 |
| | Rel. freq. | Rel. freq. | Rel. freq. | Rel. freq. |
| −5 | Nucleotide 1 | Nucleotide 2 | Nucleotide 3 | Nucleotide 4 |
| | Rel. freq. | Rel. freq. | Rel. freq. | Rel. freq. |
| −4 | Nucleotide 1 | Nucleotide 2 | Nucleotide 3 | Nucleotide 4 |
| | Rel. freq. | Rel. freq. | Rel. freq. | Rel. freq. |
| −3 | Nucleotide 1 | Nucleotide 2 | Nucleotide 3 | Nucleotide 4 |
| | Rel. freq. | Rel. freq. | Rel. freq. | Rel. freq. |
| −2 | Nucleotide 1 | Nucleotide 2 | Nucleotide 3 | Nucleotide 4 |
| | Rel. freq. | Rel. freq. | Rel. freq. | Rel. freq. |
| −1 | Nucleotide 1 | Nucleotide 2 | Nucleotide 3 | Nucleotide 4 |
| | Rel. freq. | Rel. freq. | Rel. freq. | Rel. freq. |
| 4 | Nucleotide 1 | Nucleotide 2 | Nucleotide 3 | Nucleotide 4 |
| | Rel. freq. | Rel. freq. | Rel. freq. | Rel. freq. |
| 5 | Nucleotide 1 | Nucleotide 2 | Nucleotide 3 | Nucleotide 4 |
| | Rel. freq. | Rel. freq. | Rel. freq. | Rel. freq. |
| 6 | Nucleotide 1 | Nucleotide 2 | Nucleotide 3 | Nucleotide 4 |
| | Rel. freq. | Rel. freq. | Rel. freq. | Rel. freq. |
| 7 | Nucleotide 1 | Nucleotide 2 | Nucleotide 3 | Nucleotide 4 |
| | Rel. freq. | Rel. freq. | Rel. freq. | Rel. freq. |
| 8 | Nucleotide 1 | Nucleotide 2 | Nucleotide 3 | Nucleotide 4 |
| | Rel. freq. | Rel. freq. | Rel. freq. | Rel. freq. |
| 9 | Nucleotide 1 | Nucleotide 2 | Nucleotide 3 | Nucleotide 4 |
| | Rel. freq. | Rel. freq. | Rel. freq. | Rel. freq. |

51. The method or the organism according to any one of the preceding items wherein the organism is barley, and wherein the consensus matrix is:

| | |
|---|---|
| H. vulgare | Nucleotide |
| Position | Relative frequency of the nucleotide |

| | | | | |
|---|---|---|---|---|
| −6 | G | C | A | T |
| | 33.6 | 24.8 | 22.6 | 19.1 |
| −5 | C | G | A | T |
| | 36.2 | 22.7 | 21.0 | 20.0 |
| −4 | G | C | A | T |
| | 27.8 | 27.7 | 27.4 | 17.2 |
| −3 | G | A | C | T |
| | 42.4 | 26.3 | 18.6 | 12.7 |
| −2 | C | A | G | T |
| | 44.2 | 27.2 | 16.3 | 12.3 |
| −1 | C | G | A | T |
| | 38.2 | 34.3 | 19.0 | 8.5 |
| 4 | G | C | A | T |
| | 55.3 | 16.5 | 16.1 | 12.0 |
| 5 | C | A | G | T |
| | 43.5 | 24.8 | 17.0 | 14.6 |
| 6 | G | C | T | A |
| | 42.0 | 24.5 | 19.1 | 14.4 |
| 7 | G | A | C | T |
| | 33.8 | 25.7 | 22.4 | 18.2 |
| 8 | C | T | A | G |
| | 33.8 | 22.4 | 21.9 | 21.8 |
| 9 | C | G | T | A |
| | 37.9 | 34.7 | 14.3 | 13.1 |

52. The method or the organism according to any one of the preceding items wherein the organism is rice, and wherein the consensus matrix is

| O. sativa | |
|---|---|
| subsp. japonica | Nucleotide |
| Position | Relative frequency of the nucleotide |

| | | | | |
|---|---|---|---|---|
| −6 | G | C | A | T |
| | 35.2 | 23.6 | 20.7 | 20.6 |
| −5 | C | G | T | A |
| | 35.8 | 22.8 | 21.1 | 20.3 |
| −4 | G | C | A | T |
| | 29.7 | 27.5 | 27.1 | 15.7 |
| −3 | G | A | C | T |
| | 43.1 | 25.6 | 16.5 | 14.8 |
| −2 | C | A | G | T |
| | 44.7 | 26.2 | 16.7 | 12.4 |
| −1 | C | G | A | T |
| | 37.5 | 33.3 | 20.3 | 8.9 |
| 4 | G | A | C | T |
| | 58.7 | 15.6 | 14.6 | 11.2 |
| 5 | C | A | G | T |
| | 43.1 | 25.3 | 17.4 | 14.1 |
| 6 | G | C | T | A |
| | 44.3 | 23.1 | 18.3 | 14.3 |
| 7 | G | A | C | T |
| | 35.0 | 25.7 | 21.1 | 18.1 |
| 8 | C | G | A | T |
| | 36.3 | 22.0 | 21.9 | 19.7 |
| 9 | G | C | T | A |
| | 37.5 | 32.7 | 15.7 | 14.1 |

53. The method or the organism according to any one of the preceding items, wherein the organism is barley and the consensus sequence comprises the nucleotide sequence from 5′ to 3′ GCGGCCATGGCGGCC (SEQ ID NO: 1).
54. The method or the organism according to any one of the preceding claims, wherein the generated or isolated variant organism carries substitution(s) of one or more nucleotide(s) in the TIS of the endogenous gene, wherein the substitution(s) to a nucleotide(s) identified as having a higher relative frequency at that position consist in a substitution(s) to a nucleotide(s) having at least 2% points, such as at least 5% points, for example at least 10% points, such as at least 15% points, for example at least 20% points, for example at least 25% points, such as at least 30% points, for instance at least 35% points, such as at least 38% points, for instance at least 40% points higher relative frequency, thereby increasing the probability of higher expression levels of the protein of interest.
55. The method or the organism according to any one of the preceding items, wherein the generated or isolated variant organism carries substitution(s) of one or more nucleotide(s) in the TIS of the endogenous gene, wherein the substitution(s) to a nucleotide(s) identified as having a lower relative frequency at that position consist in a substitution(s) to a nucleotide(s) having at least 2% points, such as at least 5% points, for example at least 10% points, such as at least 13% points, for example at least 15% points lower relative frequency, thereby increasing the probability of lower expression levels of the protein of interest.
56. The method or the organism according to any one of the preceding items, wherein the generated or isolated variant organism is a plant, wherein the substitution(s) in the gene of interest encoding the protein of interest of the plant is (are) associated with phenotypic traits and wherein the phenotypic traits are conserved over several growing seasons, preferably over 2 growing seasons, most preferably over 3 growing seasons.
57. The method or the organism according to any one of the preceding items, wherein the generated or isolated variant organism is barley, wherein the substitution is a +4 C/G substitution in a barley SUT2, preferably in the barley SUT2 gene sequence provided as SEQ ID NO: 13, wherein the phenotypic trait is the reduction of the proportion of lighter grains (15 mg to 35 mg) and an increase of the proportion of heavier grains (35 mg to 65 mg) compared to a wild-type barley, preferably compared to the parent organism.
58. The method or the organism according to any one of the preceding items, wherein the organism is barley, wherein the protein of interest is barley sucrose transporter 2 (HvSUT2), preferably in the barley SUT2 gene sequence provided as SEQ ID NO: 13, wherein the TIS of HvSUT2 in said variant comprises or consists of SEQ ID NO: 2.
59. The method or the organism according to any one of the preceding items wherein the organism is barley, wherein the protein of interest is beta-glucanase, wherein the barley beta glucanase gene preferably has the sequence provided as SEQ ID NO: 14, and wherein the TIS of the gene encoding beta-glucanase in said variant comprises or consists of SEQ ID NO: 3.
60. The method or the organism according to any one of the preceding items, wherein the substitution(s) is (are) positioned within nucleotide positions −6 to +9.
61. The method or the organism according to any one of the preceding items, wherein the substitution(s) is (are) positioned within nucleotide positions −10 to +13.
62. The method or the organism according to any one of the preceding items, wherein the organism is Aspergillus oryzae and wherein the consensus matrix is:

| Aspergillus | |
|---|---|
| oryzae | Nucleotide |
| Position | Relative frequency of the Nucleotide |

| | | | | |
|---|---|---|---|---|
| −6 | G | A | T | C |
| | 26.0 | 25.9 | 25.8 | 22.3 |
| −5 | C | T | A | G |
| | 31.9 | 30.2 | 24.2 | 13.7 |
| −4 | C | A | T | G |
| | 47.5 | 25.2 | 14.5 | 12.8 |
| −3 | A | G | C | T |
| | 61.5 | 22.1 | 8.6 | 7.7 |
| −2 | A | C | G | T |
| | 36.3 | 33.0 | 10.7 | 19.9 |
| −1 | C | A | G | T |
| | 35.9 | 31.0 | 18.8 | 14.3 |
| 4 | G | A | T | C |
| | 37.7 | 22.3 | 20.1 | 19.8 |
| 5 | C | A | T | G |
| | 46.4 | 23.5 | 15.3 | 14.8 |
| 6 | T | C | G | A |
| | 30.2 | 27.3 | 24.0 | 18.6 |
| 7 | G | A | C | T |
| | 27.4 | 25.4 | 24.9 | 22.3 |
| 8 | C | A | T | G |
| | 35.9 | 25.8 | 22.3 | 16.0 |
| 9 | C | T | A | G |
| | 36.5 | 23.6 | 21.1 | 18.8 |

63. The method or the organism according to any one of the preceding items, wherein the organism is Aspergillus oryzae and the consensus sequence comprises the nucleotide sequence from 5′ to 3′ GCCAACATGGCTGCC (SEQ ID NO: 6).
64. The method or the organism according to any one of the preceding items, wherein the organism is Aspergillus oryzae, wherein the protein of interest is citrate synthase (CS), preferably encoded by the coding sequence available as A0090102000627; Chromosome: 4; 2835276 to 2837167, NCBI GeneID 5994819, or by SEQ ID NO: 12, and wherein the TIS of CS in said variant comprises or consists of SEQ ID NO: 7.
65. The method or the organism according to any one of the preceding items, wherein the organism is Aspergillus oryzae, wherein the protein of interest is citrate synthase (CS), preferably encoded by the coding sequence available as AO090102000627; Chromosome: 4; 2835276 to 2837167, NCBI GeneID 5994819, or by SEQ ID NO: 12, wherein the TIS of CS in said variant comprises or consists of SEQ ID NO: 7, and wherein the expression of citrate synthase as measured by citric acid quantification is down regulated by at least 2%, such as at least 5%, for example at least 7.5%, such as at least 10%, for example at least 12.5%, such as at last 15%, for example at least 20%, such as at least 25%, for example at least 50%.
66. The method or the organism according to any one of the preceding items wherein the eukaryotic organism is a plant, and wherein the step of generating or isolating a variant organism carrying mutation(s) of item 1, step c), further comprises a step of random mutagenesis.
67. The method or the organism according to any one of the preceding items wherein the eukaryotic organism is a plant or an animal, and wherein the step of generating or isolating a variant organism carrying mutation(s) of item 1, step c), further comprises a step of gene editing using programmable nucleases, a CRISPR guide RNA system, a base editor, and/or a prime editor.
68. The method or the organism according to any one of the preceding items, wherein when the organism is a plant or an animal, the method does not comprise a step of sexual reproduction.
69. The method or the organism according to any one of the preceding items, wherein the organism is barley, wherein the protein of interest is barley sucrose transporter 2 (HvSUT2) of SEQ ID NO: 16, and wherein the TIS of HvSUT2 gene in said variant comprises or consists of SEQ ID NO: 2.
70. The method or the organism according to any one of the preceding items, wherein the organism is barley, wherein the protein of interest is beta-glucanase of SEQ ID NO: 17, and wherein the TIS of the gene encoding beta-glucanase in said variant comprises or consists of SEQ ID NO:3.
71. The method or the organism according to any one of the preceding items, wherein the organism is Aspergillus oryzae, wherein the protein of interest is citrate synthase (CS) of SEQ ID NO: 15, and wherein the TIS of the CS gene in said variant comprises or consists of SEQ ID NO: 7.
72. The method or the organism according to any one of the preceding items, with the proviso that when the organism is a plant or an animal, the plant or animal is not exclusively obtained by means of an essentially biological process (EBP).
73. An organism prepared by the method according to any one of the preceding items.
74. The method or the organism according to any one of the preceding items wherein the organism is a non-animal organism.

1. A method for modulating levels of a protein of interest in a eukaryotic organism of a species of interest, said method comprising the steps of:
a) obtaining the gDNA sequence of the translation initiation sequence (TIS) of a gene of interest encoding the protein of interest of the species,
b) comparing the gDNA sequence of the TIS of said gene with the relative frequency of each nucleotide in one or more positions of the TIS in said species or a highly similar species,
c) generating a variant organism carrying mutation(s) or isolating a variant organism carrying mutation(s), wherein said mutation(s) is (are) substitution(s) of one or more nucleotide(s) in the TIS of the endogenous gene,
wherein a substitution to a nucleotide identified as having a higher relative frequency at that position increases the probability of higher levels of the protein of interest, and
wherein a substitution to a nucleotide identified as having a lower relative frequency at that position increases the probability of lower levels of the protein of interest, and
with the proviso that the eukaryotic organism is not human.

2. The method according to claim 1, further comprising a step d) of quantifying the protein of interest levels of the generated or isolated variant organism and comparing it to the parent organism.

3. The method according to any one of the preceding claims, wherein the protein of interest has an enzymatic activity and wherein step d) is performed using an enzymatic activity assay for the protein of interest.

4. The method according to any one of the preceding claims, wherein the protein of interest level is increased or decreased by at least 2.5%, preferably by at least 5%, more preferably by at least 7.5% compared to the parent organism.

5. The method according to any one of the preceding claims, wherein the variant is prepared using programmable nucleases.

6. The method according to any one of claims 1 to 4, wherein the variant organism comprising the substitution(s) is isolated using a single nucleotide polymorphism identification technology comprising the steps of:
a. providing a pool comprising a plurality of said organisms of the species of interest, or reproductive parts thereof, representing a plurality of different genotypes;
b. dividing said pool into one or more sub-pools of organisms, or reproductive parts thereof, wherein each sub-pool comprises more than one copy of organisms of each genotype or reproductive parts thereof;
c. obtaining at least two random fractions of said sub-pool, wherein said fractions in theory each comprises organisms representing each genotype of said sub-pool
d. preparing gDNA samples from one fraction of each sub-pool, while maintaining the at least one fraction of each sub-pool for potential multiplication of organisms of each genotype within said sub-pool;
e. detecting said substitution(s) in said gDNA samples, thereby identifying sub-pool(s) comprising organism(s) or reproductive parts thereof comprising said substitution(s)
f. identifying from said identified sub-pool one or more individual organisms comprising said substitution(s).

7. A eukaryotic organism comprising one or more mutation(s) compared to the parent organism, wherein the mutation(s) is(are) in the translation initiation sequence (TIS) of a gene coding for a protein of interest, and wherein said mutation(s) is a(are) substitution(s) of one or more nucleotide(s) in the TIS of the endogenous gene,
wherein a substitution to a nucleotide identified as having a higher relative frequency at that position increases the probability of higher expression levels of the protein of interest, and wherein
a substitution to a nucleotide identified as having a lower relative frequency at that position increases the probability of lower expression levels of the protein of interest.

8. The organism according to claim 7 with the proviso that when the eukaryotic organism is a plant or an animal, the plant or animal is not exclusively obtained by means of an essentially biological process (EBP).

9. The method or the organism according to any one of the preceding claims, wherein the organism is a fungus, a yeast or a plant.

10. The method or the organisms according to any one of the preceding claims wherein the substitution(s) is (are) located between position −10 and +13, preferably between position −6 and +9, more preferably between position −6 and −1.

11. The method or the organism according to any one of the preceding claims, wherein the method comprises generating a consensus matrix of the species of interest, wherein the consensus matrix indicates the relative frequency of each nucleotide in each position of TIS in said species.

12. The method or the organism according to claim 11, wherein the consensus matrix is obtained by analyzing the TIS sequence of more than 100 genes, for example more than 5000, such as more than 10000, for example more than 15000 genes, such as more than 25000 genes, for example more than 30000 genes of the organism of interest, determining the relative frequency of each nucleotide A, T, G, and C at each nucleotide position of the TIS around the ATG start codon.

13. The method or the organism according to any one of the preceding claims, wherein the generated or isolated variant organism carries substitution(s) of one or more nucleotide(s) in the TIS of the endogenous gene,
wherein the substitution(s) to a nucleotide(s) identified as having a higher relative frequency at that position consist in a substitution(s) to a nucleotide(s) having at least 2% points, such as at least 5% points, for example at least 10% points, such as at least 15% points, for example at least 20% points, for example at least 25% points, such as at least 30% points, for instance at least 35% points, such as at least 38% points, for instance at least 40% points higher relative frequency, thereby increasing the probability of higher expression levels of the protein of interest.

14. The method or the organism according to any one of the preceding claims, wherein the generated or isolated variant organism carries substitution(s) of one or more nucleotide(s) in the TIS of the endogenous gene,
wherein the substitution(s) to a nucleotide(s) identified as having a lower relative frequency at that position consist in a substitution(s) to a nucleotide(s) having at least 2% points, such as at least 5% points, for example at least 10% points, such as at least 13% points, for example at least 15% points lower relative frequency, thereby increasing the probability of lower expression levels of the protein of interest.

15. The method or the organism according to any one of the preceding claims, wherein the organism is barley, wherein the protein of interest is barley sucrose transporter 2 (HvSUT2) of SEQ ID NO: 16, and wherein the TIS of HvSUT2 gene in said variant comprises or consists of SEQ ID NO: 2.

16. The method or the organism according to any one of claims 1 to 14, wherein the organism is barley, wherein the protein of interest is beta-glucanase of SEQ ID NO: 17, and wherein the TIS of the gene encoding beta-glucanase in said variant comprises or consists of SEQ ID NO: 3.


SEQUENCE LISTING (18 sequences)

SEQ ID NO: 1 - DNA, 15 bp, Hordeum vulgare
  misc_feature 1..15: note = Barley consensus sequence
  source 1..15: mol_type = genomic DNA
gcggccatggcggcc

SEQ ID NO: 2 - DNA, 15 bp, Hordeum vulgare
  misc_feature 1..15: note = TIS sequence of the barley sucrose transporter 2, LIM-14 mutant
  source 1..15: mol_type = genomic DNA
tgttcgatggcgccg

SEQ ID NO: 3 - DNA, 15 bp, Hordeum vulgare
  misc_feature 1..15: note = TIS sequence of the barley beta glucanase, LIM-19 mutant
  source 1..15: mol_type = genomic DNA
gactcaatggcgagc

SEQ ID NO: 4 - DNA, 15 bp, Hordeum vulgare
  misc_feature 1..15: note = TIS sequence of the barley beta glucanase, wild-type
  source 1..15: mol_type = genomic DNA
gacccaatggcgagc

SEQ ID NO: 5 - DNA, 15 bp, Hordeum vulgare
  misc_feature 1..15: note = TIS sequence of the barley sucrose transporter 2, wild type
  source 1..15: mol_type = genomic DNA
tgttcgatgccgccg

SEQ ID NO: 6 - DNA, 15 bp, Aspergillus oryzae
  source 1..15: mol_type = genomic DNA
gccaacatggctgcc

SEQ ID NO: 7 - DNA, 15 bp, Aspergillus oryzae
  source 1..15: mol_type = genomic DNA
ttcgacatgacttct

SEQ ID NO: 8 - DNA, 17 bp, synthetic construct
  source 1..17: mol_type = other DNA
tcgacatggcttcttcc

SEQ ID NO: 9 - DNA, 19 bp, synthetic construct
  source 1..19: mol_type = other DNA
ttcgacatgacttcttcct

SEQ ID NO: 10 - DNA, 20 bp, synthetic construct
  source 1..20: mol_type = other DNA
tcttcaattatactcagccg

SEQ ID NO: 11 - DNA, 20 bp, synthetic construct
  source 1..20: mol_type = other DNA
gaacagaagttccgattctc

SEQ ID NO: 12 - DNA, 1898 bp, Aspergillus oryzae
  source 1..1898: mol_type = genomic DNA
ttcgacatggcttcttccttgagaatcggaacttctgttcttcgatccacctctctcgccggtaagccggtcgtgcagtccgttgccttcaatggtttgcgctgctactctaccggcaaggccaaggttcgtcgtatcgtctagattgctcctgcttcttgacttgtctaactggcatgatttcgaactatgcagtccttgaaggagacattcgccgacaatctccctggcgagattgagaaggtcaagaagctcaggaagtatgcacgacatgcgaataataccttgaccatcagtccatttgctaaccacgccaatcaacactcgtagggactatggcaacaaggtcatcggcgaggtcaccctcgaccaggcctacggcggtgctcgtggtgtgaagtgcctcgtgtgggaagtatgtcatgacgcaacggaaccaaccagctgccgatgaggatttgctaattgtagttcagggttctgttttggattccgaagaaggtatccgtttccgtggatacaccgtatgtattatctcactgtttcttgtcgggatcatcacatactcatggttacatagatccccgaatgccagaagctgttacccaaggctcccggtggcgaggagcctcttcccgaaggtacaactaatcccctagaagacggttggtacatcctgctaatataccgtaggtctcttctggctgctgttgaccggcgaaattccctctgagcagcaggttcgcgatctgtctgccgagtgggctgctcgttctgacctccccaaattcatcgaggagctcattgaccgctgtcccagcactcttcaccccatggctcagttctctctggctgtcactgcccttgagcacgagtccgctttcgccaaggcctacgctaaaggtatcaacaagaaggactactggaactacaccttcgaggattccatggacctcattgccaagcttcctactatcgccgcgaagatttaccgcaacgtcttcaaggacggcaaggtcgctcctatccagaaggacaaggattactcctacaacttggccaaccagctcggctttggtaacaacaatgacttcgttgagctcatgcgtttgtacctgaccatccactctgaccatgagggtggaaacgtcagcgctcacaccacccaccttgttggtagtgctctgagctcccctatgctgtctttgtctgctggtctgaacggattggctggcccccttcacggattgtatgtttatccctcatcaaaaggcccacgagcgagaaatgaatgtaaacgaaaggatggtagagtcaatccagctaacacattaacttcaacagggcaaaccaggaagtgcttaactggctcacaaagatgaaggctgccattggtaacgacctcagtgaccaggccatcaaggactacttgtggtccactctcaacgctggccaggttgttcccggatacggtcacgctgtcctgcgtaagactgatcctcgttacgtttctcagcgcgagtttgcacttcgcaagctccctgatgaccaaatgttcaatctcgtcagccaggtctacaagattgctcctggcgtgctgactgagcatggcaagaccaagaaccccttccccaatgttgatgctgtaagtctattgcacatttttaaatcattcgattacaagccaacaaatgctgatctatcttcagcactctggtgtccttcttcagtactatggcctgactgaggccaactattacaccgtcctgttcggtgtctctcgtgctctaggtgttctgcctcagctgatcattgaccgcgcccttggtgcccccattgagcgccccaagtccttcagcacggaggcttacgccaagcttgtcggtgccaagctgtaa

SEQ ID NO: 13 - DNA, 1618 bp, Hordeum vulgare
  source 1..1618: mol_type = genomic DNA
ctcctcacatcccttccctccctctctcccaggtctctcgccggaggcacccacccggaggaggagatcaatcaaccaagaagggaaagcttgttcgatgccgccgcgccggcccaacaccggcgggggcgggggcacgtcgtcgtcggcggcgccggcgccgcgcaaggtccctctccggtcgctgctccgggcggcgtcggtggcgtgcggggtgcagttcgggtgggcgctgcagctgtccctgctgaccccgtacgtccaggagctgggcatcccgcacgccttcgccagcctggtgtggctgtgcggcccgctctccgggctcctggtgcagcccctggtgggccacctctcggaccgcatcgcgccggccaactccccgctcggccgccgccggcccttcatcgccgccggggcggcctccatcgccttcgccgtgctcaccgtcggcttctccgccgacctcggccgcctcttcggggacaacgtggtgcccggctccacacgcttcggcgccatcatcgtctacctcgtcggcttctggctgctcgacgtcggcaacaacgccacccagggcccatgccgcgccttcctcgccgacctcaccgagaatgacccgaggaggacccggatcgccaatgcctacttctcactcttcatggccctggggaacatactcgggtacgccaccggggcgtacaatggctggtacaagatattcccgttcactatcactgggtcctgtggcgtcagctgcgccaacctcaagtctgcgttcctgcttgatatcatcatcctggcgatcacgacgtacattagcgtggcgacggtgcaggacaatcctacatttggaagtgacgaggcggcgcctccaagcagccacgaagaggaagctttcctctttgagctattcgggtcgttcaaatacttcacgatgcctgtttggatggtcttgatcgtcacttctcttacttgggtcgggtggttcccttttatcctctttgacaccgactggatgggccgagagatctaccggggaagcccagagatcgtcgccgacacccaaaagtatcatgacggtgtgagaatgggctcttttggtctcatgctcaactcggtcgttctcgggatcacatctattggaatggagaagttgtgtaggaagtggggagctggacttgtatggggtgtctccaatatcatcatggctctgtgcttcgtggcgatgctcattataacatacgtggcgcagaacctggattatggacccagcggagcacctccaaccggaattgtcgccgcttccctcattgttttcacaatcttaggagcacctctgtcgatcacgtacagtataccgtatgcgatggctgcaagtcgtgttgaaaatcttgggctaggccaaggtctagcaatgggcattcttaatttatctattgtcataccacagatcatcgtgtcgctgggcagcgggccgtgggaccagctcttcggcggagggaacgcgccgtccttctgggtggctgccgcggcctcattcgtgggcgggctggtagccatcctggggctcccgcgggcccggctcgggccgaagaagaaaaccacccaacgatga

SEQ ID NO: 14 - DNA, 1105 bp, Hordeum vulgare
  source 1..1105: mol_type = genomic DNA
gagcctatataaggggcatggcctcaccatagaagaaagacgagagtcttacttgccccctccaacgcaggtagagagaaagagagagtttcgagacccaatggcgagccaaggcgttgcctccatgttcactctcgcattgcttctcggagccttcgcgtctatcccaccaagcgtggagtcgatcggggtgtgctacggcatgagcgccaacaacctgccggcggcgagcaccgtcgtgagcatgttcaagtccaacgggatcaaatcgatgcggctgtacgctcccaaccaggcggcgctgcaggccgtcggcggcacgggcatcaacgtcgtcgtcggggctcctaacgacgtcctctccaacctcgccgccagcccggcagcggccgcctcgtgggtcaagagcaacatccaggcgtaccccaaggtttccttccggtacgtctgcgtcggaaacgaggtcgccggcggcgccacccggaacctcgtcccggcaatgaagaacgtgcatggcgcgctcgtcgccgctgggctgggccacatcaaggtgaccacgtcggtgtcgcaggccatcctcggcgtgttcagcccgccctccgccgggtccttcaccggggaggcggccgcgttcatgggccccgtggtgcagttccttgcccgcaccaacgcgccgctcatggccaacatttacccgtacctggcctgggcttacaacccgagcgccatggacatgggctacgctctcttcaacgcgtccggcaccgtggtcagggacggcgcctacgggtaccagaacctgttcgacaccaccgtggacgccttctacacggccatgggcaagcacggcggctccagcgtgaagctggtggtgtcggagagcgggtggccgtcgggcggcggcacggcggcgactccggccaacgctaggttctacaaccagcacctcatcaaccacgtcgggcgcggcaccccacgccacccgggcgccatcgagacctacatcttcgccatgttcaacgagaaccagaaggacagcggcgtggagcagaactggggactcttctaccccaacatgcagcacgtctaccccatcaacttctga

SEQ ID NO: 15 - protein, 474 aa, Aspergillus oryzae
MASSLRIGTSVLRSTSLAGKPVVQSVAFNGLRCYSTGKAKSLKETFADNLPGEIEKVKKLRKDYGNKVIGEVTLDQAYGGARGVKCLVWEGSVLDSEEGIRFRGYTIPECQKLLPKAPGGEEPLPEGLFWLLLTGEIPSEQQVRDLSAEWAARSDLPKFIEELIDRCPSTLHPMAQFSLAVTALEHESAFAKAYAKGINKKDYWNYTFEDSMDLIAKLPTIAAKIYRNVFKDGKVAPIQKDKDYSYNLANQLGFGNNNDFVELMRLYLTIHSDHEGGNVSAHTTHLVGSALSSPMLSLSAGLNGLAGPLHGLANQEVLNWLTKMKAAIGNDLSDQAIKDYLWSTLNAGQVVPGYGHAVLRKTDPRYVSQREFALRKLPDDQMFNLVSQVYKIAPGVLTEHGKTKNPFPNVDAHSGVLLQYYGLTEANYYTVLFGVSRALGVLPQLIIDRALGAPIERPKSFSTEAYAKLVGAKL

SEQ ID NO: 16 - protein, 506 aa, Hordeum vulgare
MPPRRPNTGGGGGTSSSAAPAPRKVPLRSLLRAASVACGVQFGWALQLSLLTPYVQELGIPHAFASLVWLCGPLSGLLVQPLVGHLSDRIAPANSPLGRRRPFIAAGAASIAFAVLTVGFSADLGRLFGDNVVPGSTRFGAIIVYLVGFWLLDVGNNATQGPCRAFLADLTENDPRRTRIANAYFSLFMALGNILGYATGAYNGWYKIFPFTITGSCGVSCANLKSAFLLDIIILAITTYISVATVQDNPTFGSDEAAPPSSHEEEAFLFELFGSFKYFTMPVWMVLIVTSLTWVGWFPFILFDTDWMGREIYRGSPEIVADTQKYHDGVRMGSFGLMLNSVVLGITSIGMEKLCRKWGAGLVWGVSNIIMALCFVAMLIITYVAQNLDYGPSGAPPTGIVAASLIVFTILGAPLSITYSIPYAMAASRVENLGLGQGLAMGILNLSIVIPQIIVSLGSGPWDQLFGGGNAPSFWVAAAASFVGGLVAILGLPRARLGPKKKTTQR

SEQ ID NO: 17 - protein, 334 aa, Hordeum vulgare
MASQGVASMFTLALLLGAFASIPPSVESIGVCYGMSANNLPAASTVVSMFKSNGIKSMRLYAPNQAALQAVGGTGINVVVGAPNDVLSNLAASPAAAASWVKSNIQAYPKVSFRYVCVGNEVAGGATRNLVPAMKNVHGALVAAGLGHIKVTTSVSQAILGVFSPPSAGSFTGEAAAFMGPVVQFLARTNAPLMANIYPYLAWAYNPSAMDMGYALFNASGTVVRDGAYGYQNLFDTTVDAFYTAMGKHGGSSVKLVVSESGWPSGGGTAATPANARFYNQHLINHVGRGTPRHPGAIETYIFAMFNENQKDSGVEQNWGLFYPNMQHVYPINF

SEQ ID NO: 18 - DNA, 15 bp, Aspergillus oryzae
  source 1..15: mol_type = genomic DNA
ttcgacatggcttct